(19) United States
(12) Patent Application Publication
(10) Pub. No.: US 2024/0234000 A1
(43) Pub. Date: Jul. 11, 2024

(54) PERPENDICULAR SHAPE ANISOTROPY DESIGN WITH DUAL SPIN FILTERING

(71) Applicant: Samsung Electronics Co., Ltd., Suwon-si (KR)

(72) Inventors: Dmytro APALKOV, San Jose, CA (US); Roman CHEPULSKYY, Milpitas, CA (US); Jaewoo JEONG, San Jose, CA (US); FNU IKHTIAR, San Jose, CA (US); Sungchul LEE, Osan-Si (KR)

(21) Appl. No.: 18/116,836

(22) Filed: Mar. 2, 2023

Related U.S. Application Data
(60) Provisional application No. 63/437,359, filed on Jan. 5, 2023.

(51) Int. Cl. H01F 10/32; G11C 11/16; H10B 61/00; H10N 50/10; H10N 50/85

(52) U.S. Cl. CPC H01F 10/3286; G11C 11/161; H10B 61/22; H10N 50/10; H10N 50/85

(57) ABSTRACT

A perpendicular shape anisotropy magnetic tunnel junction structure includes a reference layer, a non-magnetic layer, and a free layer. The reference layer includes a first side and a second side opposite the first side. The non-magnetic spacer includes a first side and a second side. The first side of the non-magnetic spacer is on the second side of the first reference layer. The free layer includes a first side and a second side. The first side of the free layer is on the second side of the non-magnetic spacer. The free layer includes a first layer on the first side of the free layer, a second layer on the second side of the free layer and a coupling layer disposed between the first layer and the second layer. A ratio of a saturation magnetization of the second layer to a saturation magnetization of the first layer ranges from 0.2-0.8 inclusive.

### CROSS-REFERENCE TO RELATED APPLICATION

This application claims the priority benefit under 35 U.S.C. § 119(e) of U.S. Provisional Patent Application No. 63/437,359, filed on Jan. 5, 2023, the disclosure of which is incorporated herein by reference in its entirety. This application is also related to U.S. Patent Application Serial No. (Attorney Docket 1535-885) and U.S. Patent Application Serial No. (Attorney Docket 1535-887), both filed concurrently herewith, and the disclosure of both being incorporated herein by reference in their entireties.

### TECHNICAL FIELD

The subject matter disclosed herein relates to magnetic tunnel junction devices. More particularly, the subject matter disclosed herein relates to magnetic tunnel junction bit structures having perpendicular shape anisotropy features.

### BACKGROUND

Magnetic memories, particularly magnetic random access memories (MRAMs), have drawn increasing interest due to their potential for high read/write speed, excellent endurance, non-volatility and low power consumption during operation. An MRAM may store information utilizing magnetic materials as an information recording medium. One type of MRAM is a spin transfer torque random access memory (STT-MRAM). STT-MRAM utilizes magnetic junctions written at least in part by a current driven through the magnetic junction. A spin-polarized current driven through the magnetic junction exerts a spin torque on the magnetic moments in the magnetic junction. As a result, layer(s) having magnetic moments that are responsive to the spin torque may be switched to a desired state.

A conventional magnetic tunneling junction (MTJ) may be used in a conventional STT-MRAM. The conventional MTJ typically resides on a substrate. The conventional MTJ, uses conventional seed layer(s), may include capping layers and may include a conventional antiferromagnetic (AFM) layer. The conventional MTJ includes a conventional reference layer, a conventional free layer and a conventional tunneling barrier layer between the conventional pinned and free layers. A bottom contact below the conventional MTJ and a top contact on the conventional MTJ may be used to drive current through the conventional MTJ in a current-perpendicular-to-plane (CPP) direction.

The conventional reference layer and the conventional free layer are magnetic. The magnetization of the conventional reference layer is fixed, or pinned, in a particular direction. The conventional free layer has a changeable magnetization. The conventional free layer may be a single layer or include multiple layers.

To switch the magnetization of the conventional free layer, a current is driven perpendicular to plane. When a sufficient current is driven from the top contact to the bottom contact, the magnetization of the conventional free layer may switch to be parallel to the magnetization of a conventional bottom reference layer. When a sufficient current is driven from the bottom contact to the top contact, the magnetization of the free layer may switch to be antiparallel to that of the bottom reference layer. The differences in magnetic configurations correspond to different magnetoresistances and thus different logical states (e.g. a logical “0” and a logical “1”) of the conventional MTJ.

Because of their potential for use in a variety of applications, research in magnetic memories is ongoing. Mechanisms for improving the performance of MRAM are desired. For example, a lower switching current may be desired for easier and faster switching. Additionally, the magnetic moment of the free layer is desired to be thermally stable in the absence of a switching current being applied. Accordingly, what is needed is a method and system that may improve the switching and stability of the MRAM-based memories. The method and system described herein address such a need.

### SUMMARY

An example embodiment provides a memory device that may include an MTJ structure having perpendicular shape anisotropy. The MTJ structure may include a reference layer, a non-magnetic spacer and a free layer. The reference layer may include a first side and a second side that is opposite the first side of the reference layer. The non-magnetic spacer may include a first side and a second side in which the first side of the non-magnetic spacer may be on the second side of the reference layer. The free layer may include a first side and a second side in which the first side of the free layer may be on the second side of the non-magnetic spacer. The free layer may further include a first layer on the first side of the free layer, a second layer on the second side of the free layer and a coupling layer disposed between the first layer and the second layer. A ratio of a saturation magnetization Ms_{FL2} of the second layer to a saturation magnetization Ms_{FL1} of the first layer may range from 0.2 to 0.8 inclusive. In one embodiment, a thickness of the first layer may be substantially equal to a thickness of the second layer. In another embodiment, a ratio of a thickness of the free layer to a critical dimension of the MTJ structure may be between 0.5 and 5.0 inclusive, and the critical dimension of the MTJ structure may range from 4-22 nm inclusive. In still another embodiment, the first layer may include CoFeB, and the second layer may include CoFeX or CoFeBX in which X may be a diluent element including vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi). In yet another embodiment, the coupling layer may include a resistance area product ranging from 0.1 to 3.0 Ohm*μm² inclusive, and the coupling layer may include a tunnel magnetoresistance (TMR) that is greater than 30%. In one embodiment, the memory device may further include a capping layer on the second side of the free layer that may include one or more of magnesium oxide (MgO), tantalum oxide (TaO), niobium oxide (NiO), iridium oxide (IrO), sodium oxide (NaO), rhodium oxide (RhO), and osmium oxide (OsO). In another embodiment, the first layer may include a first side and a second side that is opposite the first side of the first layer in which the first layer may further include a first sublayer, a second sublayer and a first texture blocking layer disposed between the first sublayer and the second sublayer in which the first sublayer may be disposed distal to the coupling layer, the second sublayer may be disposed proximate to the coupling layer, and in which the second layer may include a first side and a second side that is opposite the second side of the second layer, the second layer may further include a third sublayer, a fourth sublayer and a second texture blocking layer disposed between the third sublayer and the fourth sublayer in which the third sublayer may be disposed proximate to the coupling layer and the fourth sublayer may be disposed distal to the coupling layer. In still another embodiment, at least one of the second sublayer and the third sublayer may include a CoFe or a Heusler material. In yet another embodiment, the first layer may include a first side and a second side that may be opposite the first side of the first layer in which the first layer may further include a first sublayer and a second sublayer, the first sublayer may be disposed proximate to the non-magnetic spacer, and the second sublayer may be disposed distal to the non-magnetic spacer, and in which the reference layer may further include a first layer on the first side of the reference layer and a second layer on the second side of the reference layer, the second layer of the reference layer may include a third sublayer and a fourth sublayer, the third sublayer may be distal to the non-magnetic spacer and the fourth sublayer may be proximate to the non-magnetic spacer. In one embodiment, at least one of the second sublayer and the third sublayer may include a CoFe or a Heusler material. In another embodiment, the memory device may further include an array of the MTJ structures.

An example embodiment provides a memory device that may include an MTJ structure having perpendicular shape anisotropy. The MTJ structure may include a reference layer, a non-magnetic spacer, and a free layer. The reference layer may include a first side and a second side that is opposite the first side of the reference layer. The non-magnetic spacer may include a first side and a second side in which the first side of the non-magnetic spacer may be on the second side of the reference layer. The free layer may include a first side and a second side in which the first side of the free layer may be on the second side of the non-magnetic spacer. The free layer may further include a first layer on the first side of the free layer, a second layer on the second side of the free layer and a coupling layer disposed between the first layer and the second layer. The saturation magnetization Ms_{FL2} of the second layer may be substantially equal to a saturation magnetization Ms_{FL1} of the first layer, and a ratio of a thickness of the first layer to a thickness of the second layer may range from 2 to 4 inclusive. In one embodiment, a ratio of a thickness of the free layer to a critical dimension of the MTJ structure may be between 0.5 and 5.0 inclusive, and the critical dimension of the MTJ structure may range from 4-22 nm inclusive. In another embodiment, the first layer may include CoFeB, and the second layer may include CoFeX or CoFeBX in which X is a diluent element that may include vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi). In still another embodiment, the coupling layer may include a resistance area product ranging from 0.1 to 3.0 Ohm*μm² inclusive, and the coupling layer may include a tunnel magnetoresistance (TMR) that is greater than 30%. In yet another embodiment, the memory device may include a capping layer on the second side of the free layer that may include one or more of magnesium oxide (MgO), tantalum oxide (TaO), niobium oxide (NiO), iridium oxide (rO), sodium oxide (NaO), rhodium oxide (RhO), or osmium oxide (OsO).

An example embodiment provides a memory device that may include an MTJ structure having perpendicular shape anisotropy. The MTJ structure may include a reference layer, a non-magnetic spacer, and a free layer. The reference layer may include a first side and a second side that is opposite the first side of the reference layer. The non-magnetic spacer may include a first side and a second side in which the first side of the non-magnetic spacer may be on the second side of the reference layer. The free layer may include a first side and a second side in which the first side of the free layer may be on the second side of the non-magnetic spacer. The free layer may further include a first layer on the first side of the free layer, a second layer on the second side of the free layer and a coupling layer disposed between the first layer and the second layer. A saturation magnetization Ms_{FL2} of the second layer may be substantially equal to a saturation magnetization Ms_{FL1} of the first layer, and a ratio of a thickness of the first layer to a thickness of the second layer may range from 2 to 4 inclusive. The first layer may include a first side and a second side that is opposite the first side of the first layer. The first layer may further include a first sublayer, a second sublayer and a first texture blocking layer disposed between the first sublayer and the second sublayer. The first sublayer may be disposed distal to the coupling layer, and the second sublayer may be disposed proximate to the coupling layer. The second layer may include a first side and a second side that is opposite the second side of the second layer. The second layer may further include a third sublayer, a fourth sublayer and a second texture blocking layer disposed between the third sublayer and the fourth sublayer. The third sublayer may be disposed proximate to the coupling layer and the fourth sublayer may disposed distal to the coupling layer. At least one of the second sublayer and the third sublayer may include a CoFe or a Heusler material. In one embodiment, a ratio of a thickness of the free layer to a critical dimension of the MTJ structure may be between 0.5 and 5.0 inclusive, and the critical dimension of the MTJ structure may range from 4-22 nm inclusive.

An example embodiment provides a memory device that may include an MTJ structure having perpendicular shape anisotropy. The MTJ structure may include a reference layer, a non-magnetic spacer, and a free layer. The reference layer may include a first side and a second side that is opposite the first side of the reference layer. The reference layer may further include a first layer on the first side of the reference layer and a second layer on the second side of the reference layer. The second layer of the reference layer may include a first sublayer and a second sublayer, and the second sublayer may be distal to the first side of the reference layer and proximate to the second side of the reference layer. The non-magnetic spacer may include a first side and a second side in which the first side of the non-magnetic spacer may be on the second side of the reference layer. The free layer may include a first side and a second side in which the first side of the free layer may be on the second side of the non-magnetic spacer. The free layer may further include a first layer on the first side of the free layer, a second layer on the second side of the free layer and a coupling layer disposed between the first layer and the second layer. A saturation magnetization Ms_{FL2} of the second layer may be substantially equal to a saturation magnetization Ms_{FL1} of the first layer, and a ratio of a thickness of the first layer to a thickness of the second layer may range from 2 to 4 inclusive. The first layer may include a first side and a second side that is opposite the first side of the first layer. The first layer may further include a third sublayer and a fourth sublayer in which the third sublayer may be disposed proximate to the non-magnetic spacer, and the fourth sublayer may be disposed distal to the non-magnetic spacer. At least one of the second sublayer and the third sublayer may include a CoFe or a Heusler material. In one embodiment, a ratio of a thickness of the free layer to a critical dimension of the MTJ structure may be between 0.5 and 5.0 inclusive, and the critical dimension of the MTJ structure may range from 4-22 nm inclusive.

### BRIEF DESCRIPTION OF THE DRAWING

In the following section, the aspects of the subject matter disclosed herein will be described with reference to exemplary embodiments illustrated in the figure, in which:

FIG. 1 depicts a first example embodiment of an MTJ bit structure having perpendicular shape anisotropy with a free layer having a reduced Aₑₓ according to the subject matter disclosed herein;

FIG. 2 depicts a second example embodiment of an MTJ bit structure having perpendicular shape anisotropy with a free layer having a reduced Aₑₓ according to the subject matter disclosed herein;

FIG. 3 depicts a third example embodiment of an MTJ bit structure having perpendicular shape anisotropy with a free layer having a reduced Aₑₓ according to the subject matter disclosed herein;

FIG. 4 depicts a fourth example embodiment of an MTJ bit structure having perpendicular shape anisotropy with a free layer with reduced Aₑₓ and configured for spin-orbit torque and spin transfer torque switching according to the subject matter disclosed herein;

FIG. 5 depicts a fifth example embodiment of an MTJ bit structure having perpendicular shape anisotropy with dual spin filtering according to the subject matter disclosed herein;

FIG. 5A depicts how anti-parallel-to-parallel (AP→P) switching occurs in the MTJ of FIG. 5;

FIG. 5B depicts how parallel-to-anti-parallel (P→AP) switching occurs in the MTJ of FIG. 5;

FIG. 6 depicts a sixth example embodiment of an MTJ bit structure having perpendicular shape anisotropy with dual spin filtering according to the subject matter disclosed herein;

FIG. 7 depicts a seventh example embodiment of an MTJ bit structure having perpendicular shape anisotropy with dual spin filtering according to the subject matter disclosed herein;

FIG. 8 depicts an eighth example embodiment of an MTJ bit structure having perpendicular shape anisotropy with dual spin filtering according to the subject matter disclosed herein;

FIG. 9 depicts a ninth example embodiment of an MTJ bit structure having perpendicular shape anisotropy with an asymmetric composite free layer according to the subject matter disclosed herein;

FIG. 10 depicts a tenth example embodiment of an MTJ bit structure having perpendicular shape anisotropy with an asymmetric composite free layer according to the subject matter disclosed herein;

FIG. 11 depicts an eleventh example embodiment of an MTJ bit structure having perpendicular shape anisotropy with an asymmetric composite free layer according to the subject matter disclosed herein;

FIG. 12 depicts an exemplary embodiment of a memory that may use one or more of the magnetic junction bit structures disclosed herein; and

FIG. 13 depicts an electronic device that may include a memory device that includes magnetic tunnel junction bit structures according to the subject matter disclosed herein.

### DETAILED DESCRIPTION

In the following detailed description, numerous specific details are set forth in order to provide a thorough understanding of the disclosure. It will be understood, however, by those skilled in the art that the disclosed aspects may be practiced without these specific details. In other instances, well-known methods, procedures, components and circuits have not been described in detail to not obscure the subject matter disclosed herein.

Reference throughout this specification to “one embodiment” or “an embodiment” means that a particular feature, structure, or characteristic described in connection with the embodiment may be included in at least one embodiment disclosed herein. Thus, the appearances of the phrases “in one embodiment” or “in an embodiment” or “according to one embodiment” (or other phrases having similar import) in various places throughout this specification may not necessarily all be referring to the same embodiment. Furthermore, the particular features, structures or characteristics may be combined in any suitable manner in one or more embodiments. In this regard, as used herein, the word “exemplary” means “serving as an example, instance, or illustration.” Any embodiment described herein as “exemplary” is not to be construed as necessarily preferred or advantageous over other embodiments. Additionally, the particular features, structures, or characteristics may be combined in any suitable manner in one or more embodiments. Also, depending on the context of discussion herein, a singular term may include the corresponding plural forms and a plural term may include the corresponding singular form. Similarly, a hyphenated term (e.g., “two-dimensional,” “pre-determined,” “pixel-specific,” etc.) may be occasionally interchangeably used with a corresponding non-hyphenated version (e.g., “two dimensional,” “predetermined,” “pixel specific,” etc.), and a capitalized entry (e.g., “Counter Clock,” “Row Select,” “PIXOUT,” etc.) may be interchangeably used with a corresponding non-capitalized version (e.g., “counter clock,” “row select,” “pixout,” etc.). Such occasional interchangeable uses shall not be considered inconsistent with each other.

Also, depending on the context of discussion herein, a singular term may include the corresponding plural forms and a plural term may include the corresponding singular form. It is further noted that various figures (including component diagrams) shown and discussed herein are for illustrative purpose only, and are not drawn to scale. For example, the dimensions of some of the elements may be exaggerated relative to other elements for clarity. Further, if considered appropriate, reference numerals have been repeated among the figures to indicate corresponding and/or analogous elements.

The terminology used herein is for the purpose of describing some example embodiments only and is not intended to be limiting of the claimed subject matter. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The terms “first,” “second,” etc., as used herein, are used as labels for nouns that they precede, and do not imply any type of ordering (e.g., spatial, temporal, logical, etc.) unless explicitly defined as such. Furthermore, the same reference numerals may be used across two or more figures to refer to parts, components, blocks, circuits, units, or modules having the same or similar functionality. Such usage is, however, for simplicity of illustration and ease of discussion only; it does not imply that the construction or architectural details of such components or units are the same across all embodiments or such commonly-referenced parts/modules are the only way to implement some of the example embodiments disclosed herein.

It will be understood that when an element or layer is referred to as being on, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the term “module” refers to any combination of software, firmware and/or hardware configured to provide the functionality described herein in connection with a module. For example, software may be embodied as a software package, code and/or instruction set or instructions, and the term “hardware,” as used in any implementation described herein, may include, for example, singly or in any combination, an assembly, hardwired circuitry, programmable circuitry, state machine circuitry, and/or firmware that stores instructions executed by programmable circuitry. The modules may, collectively or individually, be embodied as circuitry that forms part of a larger system, for example, but not limited to, an integrated circuit (IC), system on-a-chip (SoC), an assembly, and so forth.

Exemplary embodiments disclosed herein relate to magnetic junctions usable in magnetic devices, such as magnetic memories, and the devices using such magnetic junctions. The magnetic memories may include spin transfer torque magnetic random access memories (STT-MRAMs) and may be used in electronic devices employing nonvolatile memory. Such electronic devices include but are not limited to cellular phones, smart phones, tables, laptops and other portable and non-portable computing devices. The exemplary embodiments may also be used in other magnetic devices not configured for use as a magnetic memory. The following description is presented to enable one of ordinary skill in the art to make and use the invention and is provided in the context of a patent application and its requirements. Various modifications to the exemplary embodiments and the generic principles and features described herein will be readily apparent. The exemplary embodiments disclosed herein are mainly described in terms of particular methods and systems provided in particular implementations. The methods and systems, however, will operate effectively in other implementations. Phrases such as “exemplary embodiment,” “one embodiment” and “another embodiment” may refer to the same or different embodiments as well as to multiple embodiments. The embodiments will be described with respect to systems and/or devices having certain components. However, the systems and/or devices may include more or less components than those shown, and variations in the arrangement and type of the components may be made without departing from the scope of the invention. The exemplary embodiments will also be described in the context of particular methods having certain steps. The method and system, however, operate effectively for other methods having different and/or additional steps and steps in different orders that are not inconsistent with the exemplary embodiments. Thus, the subject matter disclosed herein is not intended to be limited to the embodiments shown, but is to be accorded the widest scope consistent with the principles and features described herein.

A bit structure for a magnetic junction is described herein. The magnetic junction includes a reference layer, a non-magnetic spacer layer and a free layer. The free layer is switchable between stable magnetic states using a current passed through the magnetic junction. The non-magnetic spacer layer is between the free layer and the reference layer. The free layer may include a soft magnetic layer, a hard magnetic layer and/or an oxide coupling layer between the hard magnetic layer and the soft magnetic layer. The soft magnetic layer may have a soft layer magnetic thermal stability coefficient of not more than 30. The hard magnetic layer may have a hard layer magnetic thermal stability coefficient of at least twice the soft layer magnetic thermal stability coefficient.

The exemplary embodiments disclosed herein are described in the context of particular methods, magnetic junctions and/or magnetic memories having certain components. One of ordinary skill in the art will readily recognize that the exemplary embodiments are consistent with the use of magnetic junctions and magnetic memories having other and/or additional components and/or other features not inconsistent with the embodiments disclosed herein. Methods and/or systems may also be described in the context of current understanding of the spin transfer phenomenon, of magnetic anisotropy, and other physical phenomenon. Consequently, one of ordinary skill in the art will readily recognize that theoretical explanations of the behavior of any method and/or system disclosed herein are made based upon this current understanding of spin transfer, magnetic anisotropy and other physical phenomena. Any methods and/or systems described herein, however, are not dependent upon a particular physical explanation. One of ordinary skill in the art will also readily recognize that any methods and/or systems described herein are described in the context of a structure having a particular relationship to the substrate. One of ordinary skill in the art, however, will readily recognize that any methods and/or systems described herein are consistent with other structures. Additionally, any methods and/or systems are described herein are described in the context of certain layers being synthetic and/or simple. One of ordinary skill in the art, however, will readily recognize that the layers could have another structure. Furthermore, any methods and/or systems described herein are described in the context of magnetic junctions and/or substructures having particular layers. One of ordinary skill in the art, however, will readily recognize that magnetic junctions and/or substructures having additional and/or different layers not inconsistent with any method and/or system described herein could also be used.

Moreover, certain components are described as being magnetic, ferromagnetic, and ferrimagnetic. As used herein, the term magnetic could include ferromagnetic, ferrimagnetic or like structures. Thus, as used herein, the term “magnetic” or “ferromagnetic” includes, but is not limited to ferromagnets and ferrimagnets. As used herein, “in-plane” is substantially within or parallel to the plane of one or more of the layers of a magnetic junction. Conversely, “perpendicular” and “perpendicular-to-plane” corresponds to a direction that is substantially perpendicular to one or more of the layers of the magnetic junction. Any methods and/or systems described herein may also be described in the context of certain alloys. Unless otherwise specified, if specific concentrations of the alloy are not mentioned, any stoichiometry not inconsistent with any method and/or system described herein may be used. The magnetic junctions are also described in the context of layers having particular thicknesses and depicted as continuous layers having a constant thickness. One of ordinary skill in the art will understand that particularly for very thin layers, one or more layer(s) of the magnetic junction may have a thickness that varies across the magnetic junction and/or may be discontinuous. Additionally, one of skill in the art will recognize when the terms “region” and “layer” are being used interchangeably.

Perpendicular Shape Anisotropy (PSA) Design with Reduced Aₑₓ

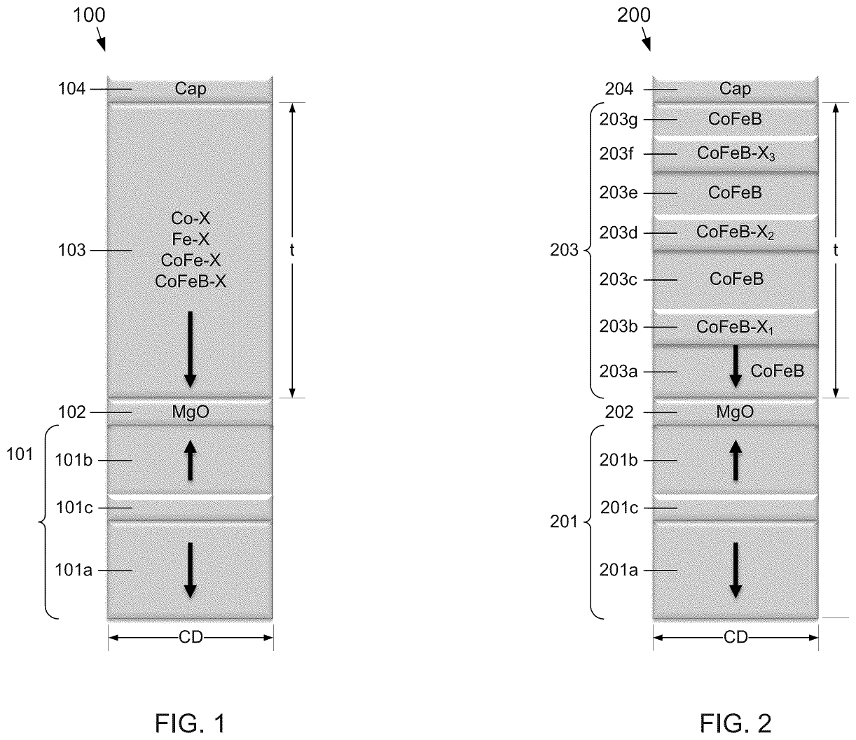

A first type of MTJ bit structure disclosed herein includes a PSA structure with a reduced Aₑₓ. As used herein, the term “perpendicular shape anisotropy” is used to describe the magnetic anisotropy of an MTJ in which the direction of easy magnetic axes is perpendicular to the layer (or film) surface. The first through the fourth example embodiments (FIGS. 1-4) disclosed herein are of this first type of MTJ bit structures. The free layer or region for a first type MTJ has a reduced Aₑₓ of about 0.5 μerg/cm that provides an increase in the Figure of Merit (FOM) (defined as the ratio of the thermal stability to the critical current (A/I_{c0})) of up to 2× over the FOM of a conventional MTJ having a PSA configuration. The FOM for a first type of MTJ bit structure may be greater than 3. The reduced Aₑₓ, which may be achieved by adding dilution elements in the free layer, may also provide a high saturation magnetization Ms so that a high energy barrier E_{b} is maintained at small critical dimensions (CDs). It should be noted that E_{b} is largely insensitive to Aₑₓ unless a very high aspect ratio (AR) (i.e., free layer thickness/CD (t/CD)) is used. In example embodiments, MTJs of the first type MTJs disclosed herein may have a CD of between about 3 and 20 nm inclusive, and may have an AR of between 0.5 and 5.0 inclusive.

FIG. 1 depicts a first example embodiment of an MTJ bit structure 100 having PSA with a free layer having a reduced Aₑₓ according to the subject matter disclosed herein. The MTJ 100 may include a reference magnetic region 101, a tunnel barrier region 102, a free magnetic region 103, and a capping region 104. The MTJ 100 may be formed on a substrate (not shown) that may include other components and/or connections (also not shown).

The reference magnetic region 101 may include at least two magnetic regions 101a and 101b that are separated by a coupling region 101c. In one embodiment, the magnetic region 101a may be formed on a substrate (not shown), and the coupling region 101c may be formed on the magnetic region 101a opposite from the substrate. The magnetic region 101b may be formed on the coupling region 101c opposite from the magnetic region 101a. Together the magnetic regions 101a and 101b and the coupling region 101c form a synthetic antiferromagnetic (SAF) region. The magnetic directions of the magnetic regions 101a and 101b are depicted in FIG. 1 by arrows within the regions 101a and 101b. The magnetic regions 101a and 101b may be referred to as being anti-parallel based on the two different direction of the arrows in the magnetic regions 101a and 101b.

The magnetic regions 101a and 101b may be formed from one or more of ferromagnetic elements nickel (Ni), iron (Fe), and/or cobalt (Co), including alloys or engineered materials with one or more of the elements palladium (Pd), platinum (Pt), nickel (Ni), and alloys thereof. The coupling region 101c may be an antiferromagnetic (AF) coupling region that includes non-ferromagnetic materials such as, for example, iridium (Ir), ruthenium (Ru), rhenium (Re), rhodium (Rh) or alloys thereof. The thickness of the non-ferromagnetic material(s) may range from 0.4 nm to 2.0 nm inclusive.

The tunnel barrier region 102 of the MTJ 100 may be formed on the magnetic region 101b opposite from the coupling region 101c. The tunnel barrier region 102 may be formed from an oxide, such as magnesium oxide (MgO). Alternatively, the tunnel barrier region 102 may be formed from Al—O, Mg—Al—O, Ti—O, Zr—O, Sc—O, Sr—O or a combination of oxides. The thickness for the tunnel barrier region 102 may be between 0.3 nm and 2.5 nm inclusive. In an alternative embodiment, the tunnel barrier region 102 may be replaced by a conducting spacer layer, such as Cu, Ge, Ag, or other similar elements or combinations (including alloys and multilayers). For the conducting spacer layer, thicknesses may be increased to a range from 0.3 nm to 200 nm inclusive.

The free magnetic region 103 may be formed on the tunnel barrier region 102 opposite from the magnetic region 101b. The free magnetic region 103 of the MTJ 100 may include one or more dilution elements that maintains Ms of the free magnetic region 103 while reducing Aₑₓ. In one embodiment, the free magnetic region 103 may be formed from Fe—X, Co—X, CoFe—X and CoFeB—X in which X is a diluent element, such as fluorine (F), strontium (Sr), nickel (Ni), rubidium (Rb), potassium (K), helium (He), platinum (Pt), iridium (Ir), manganese (Mn), ruthenium (Ru), beryllium (Be), niobium (Nb), tungsten (W), and/or molybdenum (Mo). The thickness of the free magnetic region 103 depends on the in-plane CD, and may be from 0.5*CD to 5.0*CD inclusive. In one example embodiment, the MTJ 100 may have a CD of between about 3 and 20 nm inclusive, and may have an AR of between 0.5 and 5.0 inclusive. The arrow depicted within the free magnetic region 103 indicates a magnetization direction for the free magnetic region that places the MTJ 100 in a high-resistance state (R_{H}) (antiparallel to the near magnetic region 101b or the SAF/reference magnetic region 101). When the magnetization direction of the free magnetic region 103 is in the opposite direction, the MTJ 100 is in a low-resistance state (R_{L}) (parallel to the near magnetic region 101b or the SAF/reference magnetic region 101). The other example embodiments disclosed herein similarly exhibit high- and low-resistance states based on the magnetization direction of the free magnetic region with respect to the reference magnetic region 101.

The capping region 104 may be formed on the free magnetic region 103 opposite from the tunnel barrier region 102. The capping region 104 may be a perpendicular magnetic anisotropy (PMA) controlling (i.e., increasing PMA) layer, such as one or more of magnesium oxide (MgO), tantalum oxide (TaO), nickel oxide (NiO), niobium oxide (NbO), iridium oxide (IrO), sodium oxide (NaO), rhodium oxide (RhO), or osmium oxide (OsO). The thickness of the capping region 104 may be from 0.3 nm to 2.5 nm inclusive. As the oxide capping region 104 can reduce TMR by serial parasitic resistance, the capping region 104 may be shunted on purpose by the process of forming the MTJ.

FIG. 2 depicts a second example embodiment of an MTJ bit structure 200 having PSA with a free layer having a reduced Aₑₓ according to the subject matter disclosed herein. The MTJ 200 may be considered be a variation of the MTJ 100 by having a free magnetic region formed from multiple layers. Similar to the MTJ 100, the MTJ 200 includes a free layer having a reduced Aₑₓ of about 0.5 μerg/cm. Additionally, the MTJ 200 has an increase in the FOM of up to 2× over the FOM of a conventional MTJ having a PSA configuration. The reduced Aₑₓ, which may be achieved by adding dilution elements in the free layer, may also provide a high saturation magnetization Ms so that a high energy barrier E_{b} is maintained at small CDs. Other similarities include that the MTJ 200 may have a CD of between about 3 nm to 20 nm inclusive, and may have an AR (ratio of free layer thickness t to CD) ranging from 0.5 and 5.0 inclusive.

The MTJ 200 may include a reference magnetic region 201, a tunnel barrier region 202, a free magnetic region 203, and a capping region 204. The MTJ 200 may be formed on a substrate (not shown) that may include other components and/or connections (also not shown).

The reference magnetic region 201 may include at least two magnetic regions 201a and 201b that are separated by a coupling region 201c that together form an SAF region. In one embodiment, the magnetic region 201a may be formed on a substrate (not shown), and the coupling region 201c may be formed on the magnetic region 201a opposite from the substrate. The magnetic region 201b may be formed on the coupling region 201c opposite from the magnetic region 201a. The anti-parallel magnetic directions of the magnetic regions 201a and 201b are indicated by arrows within the regions 201a and 201b. The regions 201a and 201b may be formed from one or more of ferromagnetic elements Ni, Fe and Co, including alloys or engineered materials with one or more of the elements Pd, Pt, Ni, and alloys thereof. The coupling region 201c may be an antiferromagnetic coupling region that includes non-ferromagnetic materials such as, for example, Ir, Ru, Re, Rh or alloys thereof. The thickness of the non-ferromagnetic material(s) may range from 0.4 nm to 2.0 nm inclusive.

The tunnel barrier region 202 may be formed on the magnetic region 201b opposite from the coupling region 201c. The tunnel barrier region 202 may be formed from an oxide, such as MgO. Alternatively, the tunnel barrier region 202 may be formed from Al—O, Mg—Al—O,)—O, Zr—O, Sc—O, Sr—O or a combination of oxides. The thickness of the tunnel barrier region 202 may range from 0.3 nm to 2.5 nm inclusive. In an alternative embodiment, the tunnel barrier region 202 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 203 may be formed on the tunnel barrier region 202 opposite from the magnetic region 201b. The free magnetic region 203 may include multiple layers comprising sublayers having no dilution elements alternating with sublayers having dilution elements. Together the multiple sublayers operate so that the free magnetic region 203 maintains Ms while reducing Aₑₓ. As depicted in the example embodiment of FIG. 2, the sublayers 203a, 203c, 203e and 203g may be non-dilution sublayers that may be formed from, for example, Fe, Co, CoFe and/or CoFeB. Each respective non-dilution sublayer may be formed from the same or from a different non-dilution material as other non-dilution sublayers. Dilution sublayers 203b, 203d and 203f may be formed from materials, such as Fe—X, Co—X, CoFe—X and CoFeB—X, in which X is a diluent element, such as F, Sr, Ni, Rb, K, He, Pt, Ir, Mn, Ru, Be, Nb, W, and/or Mo. The subscript for X shown in FIG. 2 indicates that each respective dilution sublayer may be formed to include the same or different diluent elements as other dilution sublayers. The thickness ratio of diluent portion may vary from 10% to 80% inclusive of the total thickness of the free magnetic region 203. It should be understood that a free magnetic region 203 may have a different number of multiple layers than the seven layers depicted in FIG. 2.

The capping region 204 may be formed on the free magnetic region 203 opposite from the tunnel barrier region 202. The capping region 204 may be a PMA-controlling (i.e., increasing PMA) layer, such as one or more of MgO, TaO, NiO, NbO, IrO, NaO, RhO, or OsO. The thickness of the capping region 204 may range from 0.3 nm to 2.5 nm inclusive. As the oxide capping region 204 can reduce TMR by serial parasitic resistance, the capping region 204 may be shunted on purpose by the process of forming the MTJ.

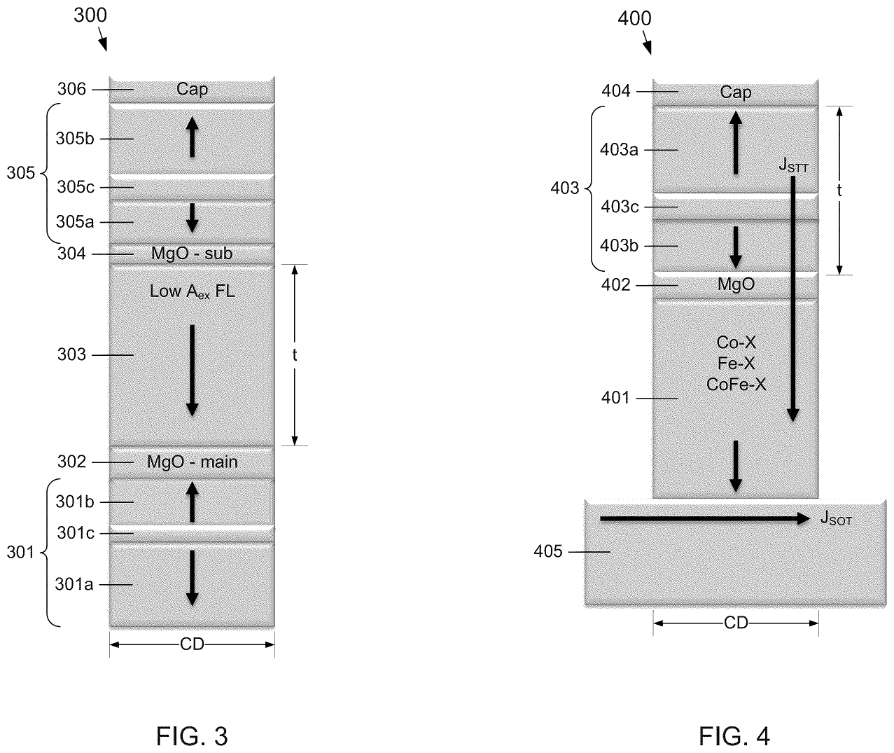

FIG. 3 depicts a third example embodiment of an MTJ bit structure 300 having PSA with a free layer having a reduced Aₑₓ according to the subject matter disclosed herein. The MTJ 300 includes two separate reference layers that form a dual MTJ design for doubling of the spin transfer torque (STT). Similar to the MTJ 100, the MTJ 300 also includes a free layer having a reduced Aₑₓ of about 0.5 μerg/cm and provides an increase in the FOM of up to 2× over the FOM of a conventional MTJ having a PSA configuration. The reduced Aₑₓ may provide a high saturation magnetization Ms so that a high energy barrier E_{b} is maintained at small CDs. The MTJ 300 may have a CD of that ranges from 3 nm to 20 nm inclusive, and may have an AR ranging from 0.5 and 5.0 inclusive.

The MTJ 300 may include a first reference magnetic region 301, a first tunnel barrier region 302, a free magnetic region 303, a secondary barrier region 304, a second reference magnetic region 305, and a capping region 306. The free magnetic region 303 may be formed between the first and second reference magnetic regions 301 and 305. The MTJ 300 may be formed on a substrate (not shown) that may include other components and/or connections (also not shown).

The first reference magnetic region 301 may include at least two magnetic regions 301a and 301b that are separated by a coupling region 301c. In one embodiment, the magnetic region 301a may be formed on a substrate (not shown). The coupling region 301c may be formed on the magnetic region 301a opposite from the substrate. The magnetic region 301b may be formed on the coupling region 301c opposite from the magnetic region 301a. Together the magnetic regions 301a and 301b and the coupling region 301c form an SAF region. The anti-parallel magnetic directions of the magnetic regions 301a and 301b are indicated by arrows within the regions 301a and 301b. The regions 301a and 301b may be formed from one or more of ferromagnetic elements Ni, Fe, and Co, including alloys or engineered materials with one or more of the elements Pd, Pt, Ni, and alloys thereof. The coupling region 301c may be an antiferromagnetic coupling region that includes non-ferromagnetic materials such as, for example, Ir, Ru, Re, Rh or alloys thereof. The thickness of the non-ferromagnetic material(s) may range from 0.4 nm to 2.0 nm inclusive.

The (main) tunnel barrier region 302 may be formed from an oxide, such as MgO. Alternatively, the tunnel barrier region 302 may be formed from Al—O, Mg—Al—O, Ti—O, Zr—O, Sc—O, Sr—O or a combination of oxides. The thickness for the tunnel barrier region 302 may range from 0.3 nm and 2.5 nm inclusive. In an alternative embodiment, the tunnel barrier region 302 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 303 may be formed on the tunnel barrier region 302 opposite from the magnetic region 301b. The free magnetic region 303 of the MTJ 300 may include one or more dilution elements that maintain Ms of the free magnetic region 303 while reducing Aₑₓ. In one embodiment, the free magnetic region 303 may include Fe—X, Co—X, CoFe—X and CoFeB—X in which X is a diluent element, such as F, Sr, Ni, Rb, K, He, Pt, Ir, Mn, Ru, Be, Nb, W, and/or Mo. The thickness of the free magnetic region 303 depends on the in-plane CD, which may vary from 0.5*CD to 5.0*CD inclusive.

The secondary barrier region 304 may be formed on the free magnetic region 303 opposite from the main barrier region 302. The secondary barrier region 304 may be formed from an oxide, such as MgO and/or aluminum oxide (Al₂O₃). Alternatively, the secondary barrier region 304 may be formed from Al—O, Mg—Al—O, Ti—O, Zr—O, Sc—O, Sr—O or a combination of oxides. The thickness of the secondary barrier region 304 may range from 0.3 nm and 2.5 nm inclusive. In an alternative embodiment, the secondary barrier region 304 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive. Alternatively, the second barrier region 304 may be formed from a metal spin-valve (SV) type spacer that provides polarization.

The second reference magnetic region 305 may be formed on the secondary barrier region 304 opposite from the free magnetic region 303. The second reference magnetic region 305 may include at least two reference magnetic regions 305a and 305b that are separated by a coupling region 305c. In one embodiment, the magnetic region 305a may be formed on the secondary barrier region 304. The coupling region 305c may be formed on the magnetic region 305a opposite from the secondary barrier region 304. The magnetic region 305b may be formed on the secondary barrier region 305c opposite from the magnetic region 305a. Together the reference magnetic regions 305a and 305b and the coupling region 305c form an SAF region. The anti-parallel magnetic directions of the magnetic regions 305a and 305b are indicated by arrows within the regions 305a and 305b. The regions 305a and 305b may be formed from one or more of ferromagnetic elements Ni, Fe, and Co, including alloys or engineered materials with one or more of the elements Pd, Pt, Ni, and alloys thereof. The coupling region 305c may be an antiferromagnetic coupling region that includes non-ferromagnetic materials such as, for example, Ir, Ru, Re, Rh or alloys thereof. The thickness of the non-ferromagnetic material(s) may range from 0.4 nm to 2.0 nm inclusive.

The capping region 306 may be formed on the second reference magnetic region 305 opposite from the secondary barrier region 304. The capping region 306 may be a PMA-controlling (i.e., increasing PMA) layer, such as one or more of MgO, TaO, NiO, NbO, IrO, NaO, RhO, or OsO. The thickness of the capping region 306 may range from 0.3 nm to 2.5 nm inclusive. As the oxide capping region 306 can reduce TMR by serial parasitic resistance, the capping region 306 may be shunted on purpose by the process of forming the MTJ.

FIG. 4 depicts a fourth example embodiment of an MTJ bit structure 400 having PSA with a free layer with reduced Aₑₓ and configured for spin-orbit torque (SOT) and spin transfer torque (STT) switching according to the subject matter disclosed herein. The MTJ 400 includes a free layer having a reduced Aₑₓ of about 0.5 μerg/cm that provides an increase in FOM of up to 2× over the FOM of a conventional MTJ having a PSA configuration. The reduced Aₑₓ may provide a high saturation magnetization Ms so that a high energy barrier E_{b} is maintained at small CDs. The MTJ 400 may have a CD of between about 3 nm to 20 nm inclusive, and may have an AR of between 0.5 and 5.0 inclusive.

The MTJ 400 may include a free magnetic region 401, a tunnel barrier layer 402, a reference magnetic region 403, and a capping region 404 that are all formed on an SOT line 405. In one embodiment, the free magnetic region 401 may be formed on the SOT line 405, and may include Fe—X, Co—X, CoFe—X and CoFeB—X in which X is a diluent element, such as F, Sr, Ni, Rb, K, He, Pt, Ir, Mn, Ru, Be, Nb, W, and/or Mo. The thickness of the free magnetic region 401 depends on the in-plane CD, which may vary from 0.5*CD to 5.0*CD inclusive.

The tunnel barrier region 402 may be formed on the free magnetic region 401. The tunnel barrier region 402 may be formed from an oxide, such as MgO. Alternatively, the tunnel barrier region 402 may be formed from Al—O, Mg—Al—O, Ti—O, Zr—O, Sc—O, Sr—O or a combination of oxides. The thickness of the tunnel barrier region 402 may range from 0.3 nm to 2.5 nm inclusive. In an alternative embodiment, the tunnel barrier region 402 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The reference magnetic region 403 may include at least two magnetic regions 403a and 403b that are separated by a coupling region 403c. The magnetic region 403a may be formed on the tunnel barrier region 402 opposite from the free magnetic region 401. The coupling region 403c may be formed on the magnetic region 403a opposite from the tunnel barrier region 402. The magnetic region 403b may be formed on the coupling region 403c opposite from the magnetic region 403a. Together the magnetic regions 403a and 403b, and the coupling region 403c form an SAF region. The anti-parallel magnetic directions of the magnetic regions 403a and 403b are indicated by arrows within the regions 403a and 403b. The regions 403a and 403b may be formed from one or more of ferromagnetic elements Ni, Fe, and/or Co, including alloys or engineered materials with one or more of the elements Pd, Pt, Ni and alloys thereof. The coupling region 403c may be an antiferromagnetic coupling region that includes non-ferromagnetic materials such as, for example, Ir, Ru, Re, Rh or alloys thereof. The thickness of the non-ferromagnetic material(s) may range from 0.4 nm to 2.0 nm inclusive.

The capping region 404 may be formed on the magnetic region 403b opposite from the coupling region 403c. The capping region 406 may be a PMA-controlling (i.e., increasing PMA) layer, such as one or more of MgO, TaO, NiO, NbO, IrO, NaO, RhO, or OsO. The thickness of the capping region 404 may range from 0.3 nm to 2.5 nm inclusive. As the oxide capping region 404 can reduce TMR by serial parasitic resistance, the capping region 404 may be shunted on purpose by the process of forming the MTJ

An SOT line 405 may include alloys of two or more materials of tungsten (W), platinum (Pt), terbium (Tb), bismuth (Bi), selenium (Se), hafnium (Hf), zirconium (Zr), silver (Ag), gold (Au), or silicon (Si) materials (e.g., W—Hf). These materials may include some oxidation at the interface between the SOT line 405 and the free magnetic region 401. Additionally, the materials used for the SOT line 405 may include multilayers (e.g., Au/Si). Topological insulators may be used, such as BiTe, BiSe, TlBiTe, TlBiSe, SbTeS, BiTeS, BiTeSe, GeSbTe, SnSbTe, GeBiTe, SnBiTe, BiSb or BiSbSe. The directions of the current densities for spin-orbit torque J_{SOT} and spin torque transfer J_{STT} are each indicated by an arrow.

Perpendicular Shape Anisotropy (PSA) Design with Dual Spin Filtering (DSF Structure)

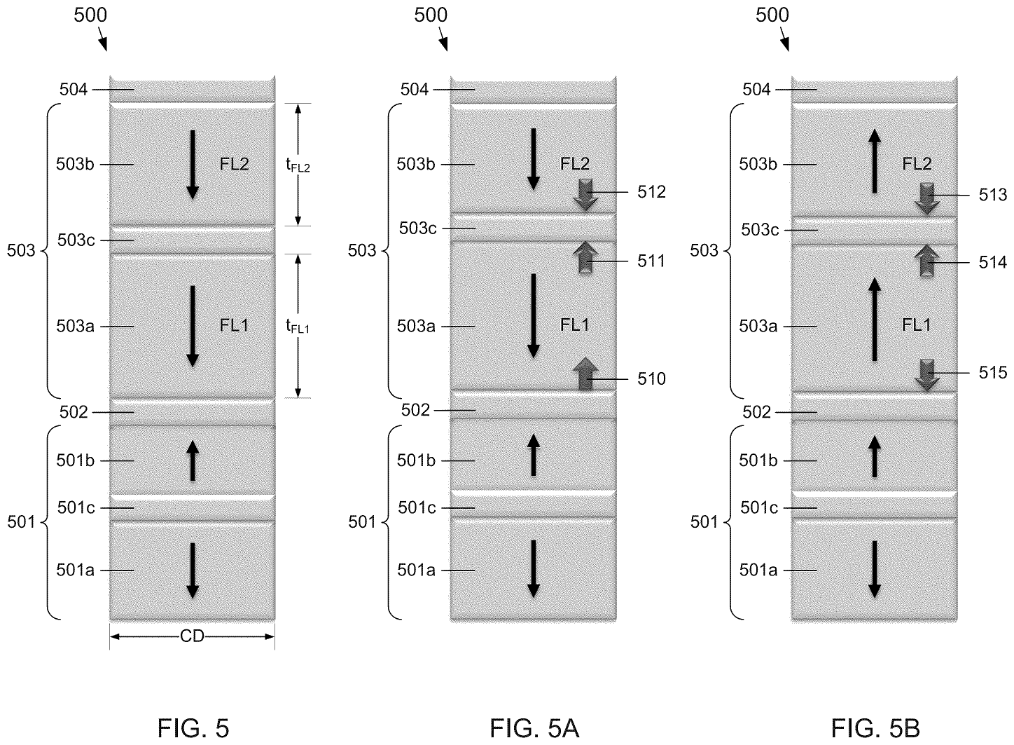

A second type of MTJ bit structure disclosed herein includes a modified PSA structure and Dual Spin Filtering (DSF). The fifth through the eight example embodiments (FIGS. 5-8) disclosed herein are of this second type of MTJ bit structures. The second type of MTJ bit structures may include a non-shunted tunnel barrier (TB) or a non-shunted spin valve (SV) that provides a spin torque transfer in which a secondary tunnel barrier (also non-shunted) also provides an STT. In one embodiment, the secondary TB may be a metallic layer that provides spin polarization. Interfacial PMA generation from the secondary TB may be optional, and the secondary TB may provide a weak coupling. MTJ structures of the second type also include a reduced switching current by up to 2× while maintaining same E_{b} by using one or more insertion layers that provide spin polarization. A free magnetic region of a second type MTJ includes a relatively softer free layer that has a relatively smaller Ms or is a thinner layer. Coupling across an insertion layer for a second type MTJ may be weak (i.e., Jₑₓ<0.5 erg/cm²). The second type MTJ structures exhibit two-stage switching because coupling across an insertion layer in the free layer may be weak. Additionally, a second type MTJ bit structure may have a CD that ranges from 4 nm to 22 nm inclusive, and may have an AR for the entire free magnetic layer (free magnetic region) stack ((t_{FL1}+t_{FL2})/CD) of between 0.5 and 5.0 inclusive.

FIG. 5 depicts a fifth example embodiment of an MTJ bit structure 500 having PSA with DSF according to the subject matter disclosed herein. The free magnetic region of the MTJ 500 may include two layers that are configured to be asymmetric in that one of the free layers is “softer” than the other free layer (i.e., easier to switch than the first free layer) by having a lower saturation magnetization Ms, either by using diluent elements or by one of the layers being thinner than the other. The MTJ 500 provides an increase in the FOM of up to 2× over a conventional MTJ having a PSA configuration.

The MTJ 500 may include a reference magnetic region 501, a main barrier region 502, a free magnetic region 503, and a capping region 504. The MTJ 500 may be formed on a substrate (not shown) that may include other components and/or connections (also not shown).

The reference magnetic region 501 may include at least two magnetic regions 501a and 501b that are separated by a coupling region 501c. The magnetic region 501a may be formed on a substrate (not shown). The coupling region 501c may be formed on the magnetic region 501a opposite from the substrate. The magnetic region 501b may be formed on the coupling region 501c opposite from the magnetic region 501a. Together the magnetic regions 501a and 501b, and the coupling region 501c form an SAF region. The anti-parallel magnetic directions of the magnetic regions 501a and 501b are indicated by arrows within the regions 501a and 501b. The regions 501a and 501b may be formed from one or more of ferromagnetic elements Ni, Fe, and/or Co, including alloys or engineered materials with one or more of the elements Pd, Pt, Ni and alloys thereof. The coupling region 501c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The main barrier region 502 may be formed on the magnetic region 501b opposite from the coupling region 501c. The main barrier region 502 may be formed from an oxide, such as MgO. Alternatively, the main barrier region 502 may be formed from Al—O, Mg—Al—O, Ti—O, Zr—O, Sc—O, Sr—O or a combination of oxides. The thickness of the main barrier region 502 may range from 0.3 nm to 2.5 nm inclusive. In an alternative embodiment, the main barrier region 502 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 503 may be formed from a first free sublayer (FL1) 503a, a second free sublayer (FL2) 503b, and a secondary tunnel barrier 503c. The first free sublayer 503a may be formed on the main barrier region 502 opposite the magnetic region 501b. The secondary tunnel barrier 503c may be formed on the first free sublayer 503a opposite from the main barrier region 502. The second free sublayer 503b may be formed on the secondary tunnel barrier 503c opposite from the first free sublayer 503a. The thickness of the first free sublayer may be approximately equal to a thickness of a second free sublayer (i.e., t_{FL1}≈t_{FL2}). In one embodiment, a ratio of t_{FL1}/t_{FL2} may range from 0.8 to 1.2 inclusive. The saturation magnetization Ms_{FL2} may be approximately half of the Ms_{FL1} so that the second free sublayer 503b is easier to switch than the first free sublayer 503a, which difference may be due to dilution of the second free sublayer 503b, as discussed below. In one embodiment, a ratio of Ms_{FL2}/Ms_{FL1} may range from 0.4 to 0.6 inclusive.

The first free sublayer 503a may be formed from CoFeB in which B is approximately 10-30%, and the ratio of Co/Fe may be approximately 0.3-2.0. The second free sublayer 503b may be formed from CoFeX or CoFeBX in which X is a diluent element, such as vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi). The thickness of the free magnetic region 503 (FL1+FL2) depends on the in-plane CD, which may vary from 0.5*CD to 5.0*CD inclusive.

The secondary tunnel barrier 503c may have a resistance-area product (RA) of approximately 0.1-3.0 Ohm*μm² and may provide a tunnel magnetoresistance of greater than 50%. In one embodiment, the secondary tunnel barrier 503c may be formed from MgO, Al₂O₃ or may be a Mg—Al—O mixture. Alternatively, the secondary tunnel barrier 503c may be formed from Al—O, Mg—Al—O, Ti—O, Zr—O, Sc—O, Sr—O or a combination of oxides. The thickness of the secondary tunnel barrier 503c may be between 0.3 nm and 2.5 nm inclusive. In an alternative embodiment, the secondary tunnel barrier 503c may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The capping region 504 may be formed on the second free sublayer 503b opposite from the secondary tunnel barrier 503c. The capping region 504 may be a PMA-controlling (i.e., increasing PMA) layer, such as one or more of MgO, TaO, NiO, NbO, IrO, NaO, RhO, or OsO. The thickness of the capping region 504 may range from 0.3 nm to 2.5 nm inclusive. As the oxide capping region 504 can reduce TMR by serial parasitic resistance, the capping region 504 may be shunted on purpose by the process of forming the MTJ.

FIG. 5A depicts how anti-parallel-to-parallel (AP→P) switching occurs in the MTJ 500. In FIG. 5A, because the coupling across the secondary TB is small, two-step switching from AP→P occurs as follows. During the first switching step, the first free sublayer 503a switches based on two STT effects that are indicated at 510 and 511. The first STT effect 510 is based on the main tunnel barrier 502. The second STT 511 effect is based on the secondary tunnel barrier 503c. The two STTs 510 and 511 add and the first free sublayer 503a is “easy” to switch. The density of the switching current may be ½*J_{c}. The second free sublayer 503b is stabilized by the secondary STT effect indicated at 512 and makes it “harder” for the second free sublayer 503b to switch. During the second switching step, the weaker free sublayer 503b switches after the first free sublayer 503a switches based on the secondary STT.

FIG. 5B depicts how parallel-to-anti-parallel (P→AP) switching occurs in the MTJ 500. In FIG. 5B, the two-step switching from P→AP begins with the second free sublayer 503b, which is weaker and switches first. The effect of the STT at 513 is not cancelled, so switching may start in the second free sublayer 503b. The STTs at 514 and 515 nearly cancel, so the first free sublayer 503a is harder to switch until sublayer 503b is switched.

For both P→AP and AP→P switching directions, the harder FL layer (first free sublayer 503a) switches by action of a combined STT effect (roughly 2× stronger) and the weaker sublayer (second free layer 503b) switches by action of a single STT.

An asymmetric MgO insertion layer (i.e., the secondary tunnel barrier 503c, which provides a thicker first free sublayer 503a) allows a reduction of switching current for both P→AP and AP→P switching directions. For P→AP switching direction, the top layer (weaker) switches first and the bottom layer switches second.

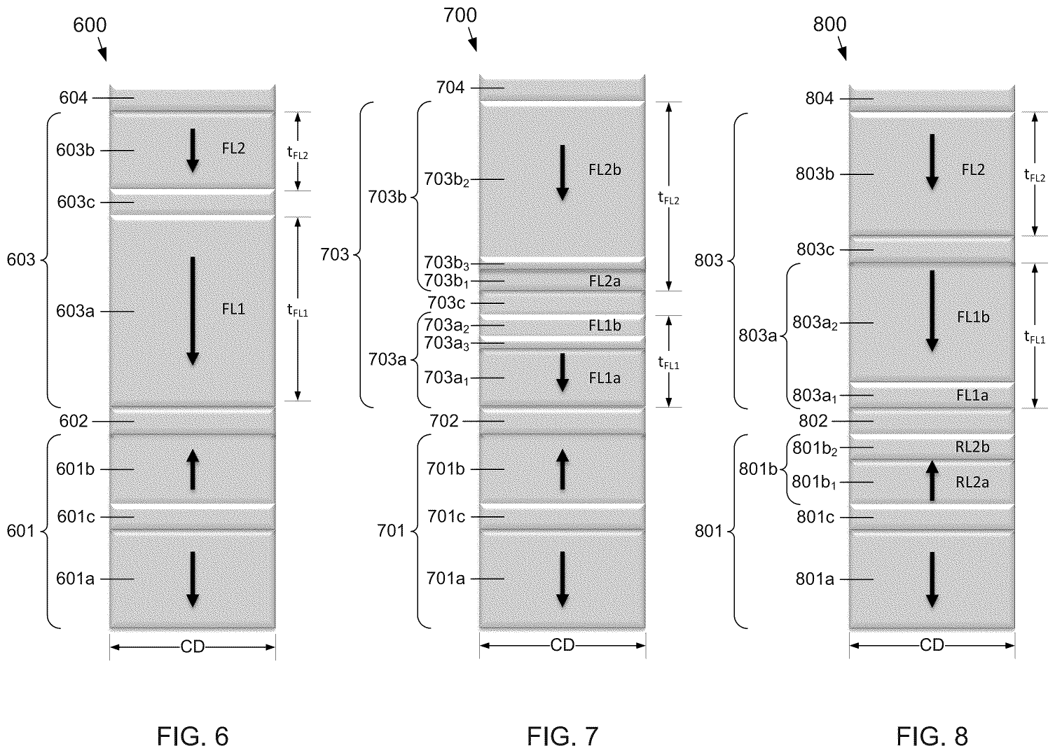

FIG. 6 depicts a sixth example embodiment of an MTJ bit structure 600 having PSA with DSF according to the subject matter disclosed herein. The MTJ 600 is similar to the structure of the MTJ 500, except that the Ms_{FL1} may be approximately equal to Ms_{FL2}, and that thickness asymmetry may be used.

The MTJ 600 may include a reference magnetic region 601, a main barrier region 602, a free magnetic region 603, and a capping region 604. The MTJ 600 may be formed on a substrate (not shown) that may include other components and/or connections (also not shown).

The reference magnetic region 601 may include at least two magnetic regions 601a and 601b that are separated by a coupling region 601c. The magnetic region 601a may be formed on a substrate (not shown). The coupling region 601c may be formed on the magnetic region 601a opposite from the substrate. The magnetic region 601b may be formed on the coupling region 601c opposite from the magnetic region 601a. Together the two magnetic regions 601a and 601b, and the coupling region 601c form an SAF region. The anti-parallel magnetic directions of the magnetic regions 601a and 601b are indicated by arrows within the regions 601a and 601b. The regions 601a and 601b may be formed from one or more of ferromagnetic elements Ni, Fe, and/or Co, including alloys or engineered materials with one or more of the elements Pd, Pt, Ni, and alloys thereof. The coupling region 601c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The main barrier region 602 may be formed on the magnetic region 601b opposite from the coupling region 601c. The main barrier region 602 may be formed from an oxide, such as MgO. Alternatively, the main barrier region 602 may be formed from Al—O, Mg—Al—O, Ti—O, Zr—O, Sc—O, Sr—O or a combination of oxides. The thickness of the main barrier region 602 may range from 0.3 nm to 2.5 nm inclusive. In an alternative embodiment, the main barrier region 602 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 603 of the MTJ 600 may include a first free sublayer (FL1) 603a, a second free sublayer (FL2) 603b, and a secondary tunnel barrier 603c. The first free sublayer 603a may be formed on the main barrier region 602 opposite the magnetic region 601b. The secondary tunnel barrier 603c may be formed on the first free sublayer 603a opposite from the main barrier region 602. The second free sublayer 603b may be formed on the secondary tunnel barrier 603c opposite from the first free sublayer 603a. The Ms_{FL1} of the first free sublayer 603a may be approximately equal to the Ms_{FL2} of the second free sublayer 603b. In one embodiment, a ratio of Ms_{FL1}/Ms_{FL2} may range from 0.8 to 1.2 inclusive. The ratio thicknesses of second free sublayer 603b to the first free sublayer 603a may be approximately 2 to 4 (i.e., t_{FL2}/t_{FL1}≈2-4) so that the second free sublayer 603b is easier to switch than the first free sublayer 603a.

The first free sublayer 603a and the second free sublayer 603b may be formed from CoFeB in which B is approximately 10-30%, and the ratio of Co/Fe may be approximately 0.3-2.0. The thickness of the free magnetic region 603 depends on the in-plane CD, which may vary from 0.5*CD to 5.0*CD inclusive.

The secondary tunnel barrier 603c may have a RA of approximately 0.1-3.0 Ohm*μm² and may provide a tunnel magnetoresistance of greater than 50%. In one embodiment, the secondary tunnel barrier 603c may be formed from MgO, Al₂O₃ or may be a Mg—Al—O mixture. Alternatively, the secondary tunnel barrier 603c may be formed from Al—O, Mg—Al—O, Ti—O, Zr—O, Sc—O, Sr—O or a combination of oxides. The thickness of the secondary tunnel barrier 603c may range from 0.3 nm to 2.5 nm inclusive. In an alternative embodiment, the secondary tunnel barrier 603c may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The capping region 604 may be formed on the second free sublayer 603b opposite from the secondary tunnel barrier 603c. The capping region 604 may be a PMA-controlling (i.e., increasing PMA) layer, such as one or more of MgO, TaO, NiO, IrO, NaO, RhO, or OsO. The thickness of the capping region 604 may range from 0.3 nm to 2.5 nm inclusive. As the oxide capping region 604 can reduce TMR by serial parasitic resistance, the capping region 604 may be shunted on purpose by the process of forming the MTJ.

FIG. 7 depicts a seventh example embodiment of an MTJ bit structure having PSA with DSF according to the subject matter disclosed herein. The seventh example embodiment MTJ 700 is similar to the fifth and sixth example embodiments MTJ 500 and MTJ 600 using PSA with DSF, but with two insertion sublayers (FL1b and FL2a) that are next to the secondary tunnel barrier (which may be a metallic spacer in certain embodiments). Additionally, an MTJ 700 includes a first insertion sublayer (FL1b) and a second insertion sublayer (FL2a). Similar to the fifth embodiment, Ms asymmetry may be used, and similar to the sixth embodiment, thickness asymmetry may be used. One or more optional Texture Blocking Layers (TBLs) may be formed in a first free sublayer and a second free sublayer to provide diffusion blocking to maintain proper composition of a Heusler material after annealing, and to provide a proper seed for the Heusler material (e.g., for insertion sublayer FL1b). The one or more TBLs may be formed from a block crystallographic texture of an insertion material (e.g., Heusler).

The MTJ 700 may include a reference magnetic region 701, a main barrier region 702, a free magnetic region 703, and a capping region 704. The MTJ 700 may be formed on a substrate (not shown) that may include other components and/or connections (also not shown).

The reference magnetic region 701 may include at least two magnetic regions 701a and 701b that are separated by a coupling region 701c. The magnetic region 701a may be formed on a substrate (not shown). The coupling region 701c may be formed on the magnetic region 701a opposite from the substrate. The magnetic region 701b may be formed on the coupling region 701c opposite from the magnetic region 701a. Together the two magnetic regions 701a and 701b, and the coupling region 701c form an SAF region. The anti-parallel magnetic directions of the magnetic regions 701a and 701b are indicated by arrows within the regions 701a and 701b. The regions 701a and 701b may be formed from one or more of ferromagnetic elements Ni, Fe, and/or Co, including alloys or engineered materials with one or more of the elements Pd, Pt, Ni, and alloys thereof. The coupling region 701c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The main barrier region 702 may be formed on the magnetic region 701b opposite from the coupling region 701c. The main barrier region 702 may be formed from an oxide, such as MgO. Alternatively, the main barrier region 702 may be formed from Al—O, Mg—Al—O, Ti—O, Zr—O, Sc—O, Sr—O or a combination of oxides. The thickness of the main barrier region 702 may range from 0.3 nm and 2.5 nm inclusive. In an alternative embodiment, the main barrier region 702 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 703 may include a first free sublayer 703a, a second free sublayer 703b, and a secondary tunnel barrier 703c. The Ms_{FL2} of the second free sublayer 703b may be approximately half of the Ms_{FL1} of the first free sublayer 703a (i.e., Ms_{FL2}≈0.5Ms_{FL1}). In one embodiment, a ratio of Ms_{FL2}/Ms_{FL1} may range from 0.4 to 0.6 inclusive. The ratio thicknesses of second free sublayer 703b to the first free sublayer 703a may be approximately 2 to 4 (i.e., t_{FL2}/t_{FL1}≈2-4). These two conditions work together so that the second free sublayer 703b is easier to switch than the first free sublayer 703a.

The first free sublayer 703a may include a free sublayer 703a₁ (FL1a), an insertion layer 703a₂ (FL1b), and an optional Texture Blocking Layer (TBL) 703a₃ (i.e., FL1=FL1a+FL1b). The free sublayer 703a₁ may be formed on the main barrier region 702 opposite from the second magnetic region 701b. The TBL 703a₃ may be formed on the free sublayer 703a₁ opposite from the main barrier region 702. When the TBL 703a₃ is present, the insertion layer 703a₂ may be formed on the TBL 703a₃ opposite from the free sublayer 703a₁. When the TBL 703a₃ is not present, the insertion layer 703a₂ may be formed on the free sublayer 703a₁ opposite the main barrier region 702.

The second free layer 703b may include an insertion layer 703b₁ (FL2a), a free sublayer 703b₂ (FL2b), and an optional TBL 703b₃, (i.e., FL2=FL2a+FL2b) The insertion layer 703b₁ may be formed on the secondary barrier layer 703c opposite from the free sublayer 703a2. The TBL 703b₃ may be formed on the insertion layer 703b₁ opposite from the secondary tunnel barrier 703c. When the TBL 703b₃ is present, the free sublayer 703b₂ may be formed on the TBL 703b₃ opposite from the insertion layer 703b₁. When the TBL 703b₃ is not present, the free sublayer 703b₂ may be formed on the insertion layer 703b₁ opposite from the secondary tunnel barrier 703c.

The first free sublayer 703a may be formed from CoFeB in which B is approximately 10-30%, and the ratio of Co/Fe may be approximately 0.3-2.0. The second free sublayer 703b may be formed from CoFeX or CoFeBX in which X is a diluent element, such as vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi). The thickness of the free magnetic region 703 (FL1+FL2) depends on the in-plane CD, which may vary from 0.5*CD to 5.0*CD inclusive.

The materials forming the layers 703a₂ and 703b₁ have a high polarization, a low damping, and may have a high Ms to increase polarization and TMR of a secondary tunnel barrier 703c, which in one embodiment may be a metal spacer. Example high-Ms materials that may be used for the layers 703a₂ and 703b₁ include CoFe and Heusler compounds and the thickness of insertion layers 703a₂ and 703b₁ may range from 0.2 nm to 2 nm inclusive.

The optional Texture Blocking Layers 703a3 and 703b₃ may be respectively formed in the first free sublayer 703a and the second free sublayer 703b to provide diffusion blocking to maintain proper composition of Heusler material after annealing, and to provide a proper seed for the Heusler compound (e.g., for FL1b). One or both of the TBLs 703a3 and 703b₃ may be used. As depicted in FIG. 7, a TBL 703a₃ may be formed in the first free layer 703a between the free layer 703a₁ and the insertion layer 703b₂. Similarly, a TBL 703b₃ may be formed in the second free layer 703b between the insertion layer 703b1 and the free layer 703b₂. Each TBL may be formed to block crystallographic texture of insertion material to increase read signal from the Heusler compound. Materials that may be used for the TBLs are usually amorphous and may include W, Ta, Mo, Nb, CoFe+X or CoFeB+X in which X may be W, Ta, Mo and/or Nb.

The secondary tunnel barrier 703c may have a RA of approximately 0.1-3.0 Ohm*μm² and may provide a tunnel magnetoresistance of greater than 50%. Alternatively, the secondary tunnel barrier 703c may be formed from MgO, Al₂O₃ or may be a Mg—Al—O mixture. In another alternative embodiment, the secondary tunnel barrier 703c may be formed from Al—O, Mg—Al—O, Ti—O, Zr—O, Sc—O, Sr—O or a combination of oxides. The thickness of the secondary tunnel barrier 703c may range from 0.3 nm to 2.5 nm inclusive. In still another alternative embodiment, the secondary tunnel barrier 703c may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The capping region 704 may be formed on the free sublayer 703b₂ opposite from the TBL 703b₃ when the TBL 703b₃ is present. When the TBL 703b₃ is not present, the capping region 704 may be formed on the free sublayer 703b₂ opposite from the free sublayer 703b₁. The capping region 704 may be a PMA-controlling (i.e., increasing PMA) layer, such as one or more of MgO, TaO, NiO, IrO, NaO, RhO, or OsO. The thickness of the capping region 704 may range from 0.3 nm to 2.5 nm inclusive. As the oxide capping region 704 can reduce TMR by serial parasitic resistance, the capping region 704 may be shunted on purpose by the process of forming the MTJ.

FIG. 8 depicts an eighth example embodiment of an MTJ bit structure 800 having PSA with DSF according to the subject matter disclosed herein. In FIG. 8, the main spacer 802 may be a metallic layer to reduce RA for small CDs. The first free layer and the second reference layer may include insertion layers to maximize TMR that may be formed from the same materials forming the insertion layers of the MTJ 700 The thickness of insertion layers may range from 0.2 nm to 2 nm.

The MTJ 800 may include a reference magnetic region 801, a main barrier region 802, a free magnetic region 803, and a capping region 804. The MTJ 800 may be formed on a substrate (not shown) that may include other components and/or connections (also not shown).

The reference magnetic region 801 may include at least two magnetic regions 801a and 801b that are separated by a coupling region 801c. The magnetic region 801a may be formed on a substrate (not shown). The coupling region 801c may be formed on the magnetic region 801a opposite from the substrate. The magnetic region 801b may be formed on the coupling region 801c opposite from the magnetic region 801a. Together the two magnetic regions 801a and 801b, and the coupling region 801c form an SAF region. The anti-parallel magnetic directions of the magnetic regions 801a and 801b are indicated by arrows within the regions 801a and 801b. The regions 801a and 801b may be formed from one or more of ferromagnetic elements Ni, Fe, and/or Co, including alloys or engineered materials with one or more of the elements Pd, Pt, Ni, and alloys thereof. The coupling region 801c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The second reference magnetic region 801b may include an insertion layer 801b₂ so that the second reference magnetic region 801b is formed from a reference layer 801b₁ (RL2a) and the insertion layer 801b₂ (RL2b). The insertion layer 801b₂ may be formed on the reference layer 801b₁ opposite from the coupling region 801c. The insertion layer 801b₂ may be formed from a material having a high polarization, a low damping, and may have a high Ms to increase polarization and TMR. Example high-Ms materials that may be used may include CoFe and Heusler compounds. The thickness of insertion layer 801b₂ may range from 0.2 nm to 2 nm inclusive.

The main barrier region 802 may be formed on the insertion layer 801b₂ opposite from the reference layer 801b₁. The main barrier region 802 may be formed from an oxide, such as MgO. Alternatively, the main barrier region 802 may be formed from Al—O, Mg—Al—O, Ti—O, Zr—O, Sc—O, Sr—O or a combination of oxides. The thickness of the main barrier region 802 may range from 0.3 nm and 2.5 nm inclusive. In an alternative embodiment, the main barrier region 802 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region 803 may include a first free layer 803a, a second free layer (FL2) 803b and a secondary tunnel barrier 803c. The Ms_{FL2} of the second free layer 803b may be approximately half of the Ms_{FL1}′ of the first free layer 803a (i.e., Ms_{FL2}≈0.5Ms_{FL1}). In one embodiment, a ratio of Ms_{FL2}/Ms_{FL1} may range from 0.4 to 0.6 inclusive. The thickness of second free layer 803b may be approximately equal to the thickness of the first free layer 803a (i.e., t_{FL2}≈t_{FL1}). In one embodiment, a ratio of t_{FL2}/t_{FL1} may range from 0.8 to 1.2 inclusive. These two conditions work together so that the second free layer 803b is easier to switch than the first free layer 803a.

The first free sublayer 803a may include an insertion layer 803a₁ (FL1a) and a free sublayer 803a₂ (FL1b) (i.e., FL1=FL1a+FL1b). The insertion layer 803a₁ may be formed on the main barrier region 802 opposite from the insertion layer 801b₂. The free sublayer 803a₂ may be formed on the insertion layer 803a₁ opposite from the main barrier region 802. The thickness of insertion layer 803b₁ may range from 0.2 nm to 2 nm. The insertion layer 803a₁ may be formed from a material having a high polarization, a low damping, and may have a high Ms to increase polarization and TMR. Example high-Ms materials that may be used include CoFe and Heusler compounds. The first free layer 803a₂ may be formed from CoFeB in which B is approximately 10-30%, and the ratio of Co/Fe may be approximately 0.3-2.0.

The secondary tunnel barrier 803c may be formed on the first free layer 803a₂ opposite from the insertion layer 803a₁. The secondary tunnel barrier 803c may have a RA of approximately 0.1-3.0 Ohm*μm² and may provide a tunnel magnetoresistance of greater than 50%. Alternatively, the secondary tunnel barrier 803c may be formed from MgO, Al₂O₃ or may be a Mg—Al—O mixture. In another alternative embodiment, the secondary tunnel barrier 803c may be formed from Al—O, Mg—Al—O, Ti—O, Zr—O, Sc—O, Sr—O or a combination of oxides. The thickness of the secondary tunnel barrier 803c may range from 0.3 nm and 2.5 nm inclusive. In still another alternative embodiment, the secondary tunnel barrier 803c may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The second free sublayer 803b may be formed on the secondary tunnel barrier 803c opposite from the first free sublayer 803a2. The second free layer 803b may be formed from CoFeX or CoFeBX in which X is a diluent element, such as vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi). The thickness of the free magnetic region 803 (FL1+FL2) depends on the in-plane CD, which may vary from 0.5*CD to 5.0*CD inclusive.

The capping region 804 may be formed on the second free sublayer 803b opposite from the secondary tunnel barrier 803c. The capping region 804 may be a PMA-controlling (i.e., increasing PMA) layer, such as one or more of MgO, TaO, NiO, IrO, NaO, RhO, or OsO. The thickness of the capping region 804 may range from 0.3 nm to 2.5 nm inclusive. As the oxide capping region 804 can reduce TMR by serial parasitic resistance, the capping region 804 may be shunted on purpose by the process of forming the MTJ.

Perpendicular Shape Anisotropy Design with Asymmetric Composite FL (ACFL)

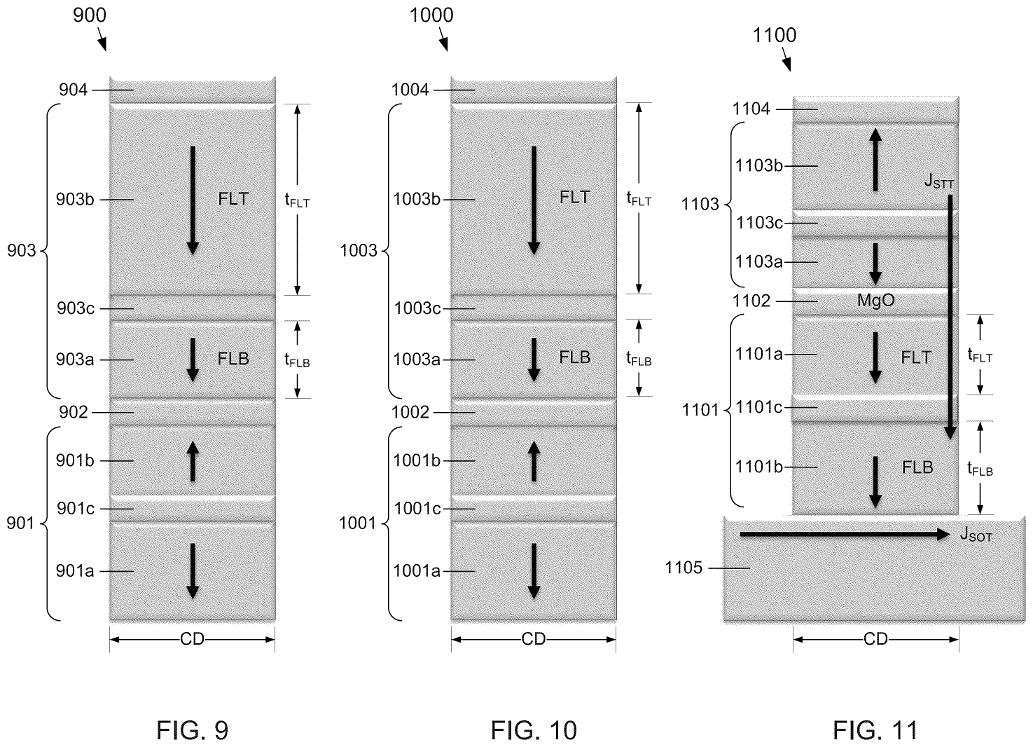

A third type of MTJ bit structure disclosed herein includes PSA with an asymmetric composite free layer (ACFL). The ninth through eleventh embodiments (FIGS. 9-11) are third type MTJ bit structures. The ACFL may provide an improved FOM by around 2× over the FOM of a conventional MTJ. A “softer” free layer in which it is easier to change magnetic direction of the layer is next to a main tunnel barrier and includes a coupled bilayer of the first free layer and the second free layer. The first free layer FL1 is softer (easier to switch) and has smaller Ms or thickness than the second free layer. The coupling layer provides ferromagnetic coupling that ranges between 0.5-1.5 erg/cm² inclusive. A third type MTJ bit structure may have a CD that ranges from 4 nm to 22 nm inclusive, and may have an AR for the entire free magnetic layer (free magnetic region) stack ((t_{FLB}+t_{FLT})/CD) of between 1.0 and 2.5 inclusive.

FIG. 9 depicts a ninth example embodiment of an MTJ bit structure 900 having PSA with ACFL according to the subject matter disclosed herein. The MTJ 900 may include a reference magnetic region 901, a main barrier region 902, a free magnetic region 903, and a capping region 904. The MTJ 900 may be formed on a substrate (not shown) that may include other components and/or connections (also not shown).

The reference magnetic region 901 may include at least two magnetic regions 901a and 901b that are separated by a coupling region 901c. The magnetic region 901a may be formed on a substrate (not shown). The coupling region 901c may be formed on the magnetic region 901a opposite from the substrate. The magnetic region 901b may be formed on the coupling region 901c opposite from the magnetic region 901a. Together the two magnetic regions 901a and 901b, and the coupling region 901c form an SAF region. The anti-parallel magnetic directions of the magnetic regions 901a and 901b are indicated by arrows within the regions 901a and 901b. The regions 901a and 901b may be formed from one or more of ferromagnetic elements Ni, Fe, and/or Co, including alloys or engineered materials with one or more of the elements Pd, Pt, Ni, and alloys thereof. The coupling layer 901c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The main barrier region 902 may be formed on the magnetic region 901b opposite from the coupling region 901c. The main barrier region 902 may be formed from an oxide, such as MgO. Alternatively, the main barrier region 902 may be formed from Al—O, Mg—Al—O, Ti—O, Zr—O, Sc—O, Sr—O or a combination of oxides. The thickness of the main barrier region 902 may range from 0.3 nm and 2.5 nm inclusive. In an alternative embodiment, the main barrier region 902 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The free magnetic region of the MTJ 900 may include a first free sublayer (FLB) 903a, a second free sublayer (FLT) 903b and a secondary coupling layer 903c. The first free sublayer 903a may be formed on the main barrier region 902 opposite the magnetic region 901b. The secondary coupling layer 903c may be formed on the first free sublayer 903a opposite from the main barrier region 902. The second free sublayer 903b may be formed on the secondary coupling layer 903c opposite from the first free sublayer 903a. The ratio of Mst_{FL2} of the second free sublayer 903b to the Mst_{FL1} of the first free sublayer may be approximately 2-5, which ensures that the first free sublayer 903a is easier to switch than the second free sublayer 903b. The first free sublayer 903a may be formed from Co—Fe—X or Co—Fe—B—X in which X is a diluent element, such as V, Mo, Ti, Zr, Hf, Al, Nb, Ta, Cr, Rh or Bi. The second free sublayer 903b may be formed from Co—Fe or Co—Fe—B in which B is approximately 10-30%, and the ratio of Co/Fe may be approximately 0.3-2.0. In one embodiment, a ratio of t_{FLT}/t_{FLB} may range from 4.0 to 1.0 inclusive.

In one embodiment, the secondary coupling layer 903c may be formed from a weakly ferromagnetic material, such as Co—Fe—X in which X may be Mo, W, Ta, Cr, Hf, Nb, Ti, Zr and Ni. In another embodiment, the secondary coupling layer 903c may be formed from a thin oxide layer, such as MgO, AlO, MAO (Magnesium Aluminum Oxide) or NiO. In still another embodiment, the secondary coupling layer 903c may be formed from a thin non-magnetic layer having a high melting temperature or a low diffusion, such as W, Mo or Ta. A desired degree of coupling provided by the secondary coupling layer 903c may range from 0.5-3.0 erg/cm² inclusive.

Selection of the insertion position of the secondary coupling layer 903c within the free magnetic region 903 may be characterized by the ratio t_{FLB}/(t_{FLB}+t_{FLT}) in which if the ratio is selected to be equal to 0.5, the insertion position is in the middle of the free magnetic region 903. If the ratio is selected to be less than 0.5, the insertion position of the secondary coupling layer 903c is closer to the main barrier region 902 (i.e., the bottom sublayer of the free layer 903 is thinner). If the ratio is selected to be greater than 0.5, the insertion position of the secondary coupling layer 903c is closer to the capping region 904 (i.e., the bottom sublayer of the free layer 903 is thicker).

The capping region 904 may be formed on the second free sublayer 903b opposite from the secondary coupling layer 903c. The capping region 904 may be a PMA-controlling (i.e., increasing PMA) layer, such as one or more of MgO, TaO, NiO, IrO, NaO, RhO, or OsO. The thickness of the capping region 904 may range from 0.3 nm to 2.5 nm inclusive. As the oxide capping region 904 can reduce TMR by serial parasitic resistance, the capping region 904 may be shunted on purpose by the process of forming the MTJ.

FIG. 10 depicts a tenth example embodiment of an MTJ bit structure 1000 having PSA with ACFL according to the subject matter disclosed herein. The MTJ 1000 is similar to the MTJ 900 in FIG. 9, but differs by the materials of the free magnetic region including Heusler compounds, such as but not limited to Co₂FeAl. Additionally, the main tunnel barrier 902 of the MTJ 900 is replaced by a metallic spacer that provides high polarization (and GMR) in combination with Heusler materials for the first and second free layers of the free magnetic region.

The MTJ 1000 may include a reference magnetic region 1001, a main barrier region 1002, a free magnetic region 1003, and a capping region 1004. The MTJ 1000 may be formed on a substrate (not shown) that may include other components and/or connections (also not shown).

The reference magnetic region 1001 may include at least two magnetic regions 1001a and 1001b that are separated by a coupling region 1001c. The magnetic region 1001a may be formed on a substrate (not shown). The coupling region 1001c may be formed on the magnetic region 1001a opposite from the substrate. The magnetic region 1001b may be formed on the coupling region 1001c opposite from the magnetic region 1001a. Together the two magnetic regions 1001a and 1001b, and the coupling region 1001c form an SAF region. The anti-parallel magnetic directions of the magnetic regions 1001a and 1001b are indicated by arrows within the regions 1001a and 1001b. The regions 1001a and 1001b may be formed from one or more of ferromagnetic elements Ni, Fe, and/or Co, including alloys or engineered materials with one or more of the elements Pd, Pt, Ni, and alloys thereof. The coupling layer 1001c may be an AF coupling region that includes non-ferromagnetic materials, such as Ir, Ru, Re, Rh or alloys thereof. The thickness of the non-ferromagnetic material(s) may range from 0.4 nm to 2.0 nm inclusive.

The main barrier region 1002 may be formed on the magnetic region 1001b opposite from the coupling region 1001c. The main barrier region 1002 may be formed from a metallic spacer that provides high polarization and GMR in combination with Heusler materials forming the free magnetic region 1003.

The free magnetic region 1003 of the MTJ 1000 may include a first free sublayer (FLB) 1003a, a second free sublayer (FLT) 1003b and a secondary coupling layer 1003c. The first free sublayer 1003a may be formed on the main barrier region 1002 opposite the magnetic region 1001b. The secondary coupling layer 1003c may be formed on the first free sublayer 1003a opposite from the main barrier region 1002. The second free sublayer 1003b may be formed on the secondary coupling layer 1003c opposite from the first free sublayer 1003a. The ratio of Mst_{FL2} of the second free layer 1003b to the Mst_{FL1} of the first free layer may be approximately 2-5, which ensures that the first free layer (FLB) 1003a is easier to switch than the second free layer (FLT) 1003b. In one embodiment, the first free layer 1003a may be formed from Co—Fe—X or Co—Fe—B—X in which X is a diluent element, such as V, Mo, Ti, Zr, Hf, Al, Nb, Ta, Cr, Rh or Bi, and the second free layer 1003b may be formed from high Ms Heusler compounds. The thickness of the free magnetic region 1003 (FLB+FLT) depends on the in-plane CD, which may vary from 0.5*CD to 5.0*CD inclusive. In an alternative embodiment, the first free layer 1003a may be formed from high Ms Heusler compounds, and the second free layer 1003b may be formed from Co—Fe or Co—Fe—B in which B is approximately 10-30%, and the ratio of Co/Fe may be approximately 0.3-2.0. For this alternative embodiment, the thickness of the free magnetic region 1003 (FLB+FLT) depends on the in-plane CD, which may vary from 0.5*CD to 5.0*CD inclusive. Additionally, the thickness t_{FLT} of the second free layer 1003b may range from 1.2 to 5.0 (inclusive) times thicker than the thickness t_{FLB} of the first free layer 1003a.

The secondary coupling layer 1003c may be a weakly ferromagnetic material, such as Co—Fe—X in which X may be Mo, W, Ta, Cr, Nb. Alternatively, the coupling layer may be MgO, AlO, MAO or NiO. Still another alternative provides that the coupling layer may have a high melting temperature or low diffusion, such as tungsten (W), molybdenum (Mo) or tantalum (Ta). The coupling range of the secondary coupling layer 1003c may be between 0.5 and 3.0 erg/cm² inclusive.

The capping region 1004 may be formed on the second free sublayer 1003b opposite from the secondary coupling layer 1003c. The capping region 1004 may be a PMA-controlling (i.e., increasing PMA) layer, such as one or more of MgO, TaO, NiO, IrO, NaO, RhO, or OsO. The thickness of the capping region 1004 may range from 0.3 nm to 2.5 nm inclusive. As the oxide capping region 1004 can reduce TMR by serial parasitic resistance, the capping region 1004 may be shunted on purpose by the process of forming the MTJ.

FIG. 11 depicts an eleventh example embodiment of an MTJ bit structure 1100 having PSA with ACFL according to the subject matter disclosed herein. The MTJ 1100 has similarities to the MTJ 900 in FIG. 9, and includes an SOT line on which the reference magnetic region may be formed.

The MTJ 1100 may include a free magnetic region 1101, a main barrier region 1102, a reference magnetic region 1103, and a capping region 1104 that are all formed on an SOT line 1105. The free magnetic region 1101 of the MTJ 1100 may include a first free sublayer (FLB) 1101a, a second free sublayer (FLT) 1101b and a secondary coupling layer 1101c. The first free sublayer 1101a may be formed on the SOT line 1105. The secondary coupling layer 1101c may be formed on the first free sublayer 1101a opposite from the SOT line 1105. The second free sublayer 1101b may be formed on the secondary coupling layer 1101c opposite from the first free sublayer 1101a. The ratio of Mst_{FLT} of the second free sublayer 1101b to the Mst_{FLB} of the first free sublayer 1101a may be approximately 2-5, which ensures that the first free sublayer 1101a is easier to switch than the second free sublayer 1101b.

The first free sublayer 1101a may be formed from Co—Fe—X or Co—Fe—B—X in which X is a diluent element, such as V, Mo, Ti, Zr, Hf, Al, Nb, Ta, Cr, Rh or Bi. The second free sublayer 1101b may be formed from Co—Fe or Co—Fe—B in which B is approximately 10-30%, and the ratio of Co/Fe may be approximately 0.3-2.0. The thickness of the free magnetic region 1101 (FLB+FLT) depends on the in-plane CD, which may range from 0.5*CD to 5.0*CD inclusive. Additionally, the thickness t_{FLT} of the second free sublayer 1101b may range from 1.5 to 5.0 times thicker than the thickness t_{FL}B of the first free sublayer 1101a.

In one embodiment, the secondary coupling layer 1101c may be formed from a weakly ferromagnetic material, such as Co—Fe—X in which X may be Mo, W, Ta, Cr, Nb, Ti, Hf, Zr, and Ni. In another embodiment, the secondary coupling layer 1101c may be formed from a thin oxide layer, such as MgO, AlO, MAO or NiO. In still another embodiment, the secondary coupling layer 1101c may be formed from a thin non-magnetic layer having a high melting temperature or a low diffusion, such as tungsten (W), molybdenum (Mo) or tantalum (Ta). A desired degree of coupling provided by the secondary coupling layer 1101c may be between 0.5-3.0 erg/cm² inclusive.

The main barrier region 1102 may be formed on the second free sublayer 1101b opposite from the secondary coupling layer 1101c. The main barrier region 1102 may be formed from an oxide, such as MgO. Alternatively, the main barrier region 1102 may be formed from Al—O, Mg—Al—O, Ti—O, Zr—O, Sc—O, Sr—O or a combination of oxides. The thickness of the main barrier region 1102 may range from 0.3 nm to 2.5 nm inclusive. In an alternative embodiment, the main barrier region 1102 may be replaced by a conducting spacer layer, such as Cu, Ge, Ag, or other similar elements or combinations (including alloys and multilayers). For the conducting spacer layer, thicknesses may be increased and may range from 0.3 nm to 200 nm inclusive.

The reference magnetic region 1103 may include at least two magnetic regions 1103a and 1103b that are separated by a coupling layer 1103c. The magnetic region 1103a may be formed on the main barrier region 1102 opposite from the second free sublayer 1101b. The coupling region 1103c may be formed on the magnetic region 1103a opposite from the main barrier region 1102. The magnetic region 1103b may be formed on the coupling region 1103c opposite from the magnetic region 1103a. Together form the two magnetic regions 1103a and 1103b, and the coupling region 1103c an SAF region. The anti-parallel magnetic directions of the magnetic regions 1103a and 1103b are indicated by arrows within the regions 1103a and 1103b. The regions 1103a and 1103b may be formed from one or more of ferromagnetic elements Ni, Fe, and/or Co, including alloys or engineered materials with one or more of the elements Pd, Pt, Ni, and alloys thereof. The coupling layer 1103c may be an antiferromagnetic coupling region that includes non-ferromagnetic materials, such as Ir, Ru, Re, Rh or alloys thereof. The thickness of the non-ferromagnetic material(s) may range from 0.4 nm to 2.0 nm inclusive.

The capping region 1104 may be formed on the second magnetic region 1103b opposite from the coupling region 1103c. The capping region 1104 may be a PMA-controlling (i.e., increasing PMA) layer, such as one or more of MgO, TaO, NiO, IrO, NaO, RhO, or OsO. The thickness of the capping region 1104 may range from 0.3 nm to 2.5 nm inclusive. As the oxide capping region 1104 can reduce TMR by serial parasitic resistance, the capping region 1104 may be shunted on purpose by the process of forming the MTJ.

Materials that may be used to form the SOT line 1105 may include alloys of two or more materials of W, Pt, Tb, Bi, Se, Hf, Zr, Ag, Au, and Si materials (e.g., W—Hf) in which the materials used have some oxidation at the interface. Additionally, the previously listed materials may be formed to include multilayers (e.g., Au/Si). The topological insulators that may be used for the SOT line 1105 may be formed from (BiTe, BiSe, TlBiTe, TlBiSe, SbTeS, BiTeS, BiTeSe, GeSbTe, SnSbTe, GeBiTe, SnBiTe, BiSb, or BiSbSe).

Table 1 sets forth improvements for MTJ bit structure using PSA with DSF (embodiments 5-8 (FIGS. 5-8)) and for MTJ bit structures using PSA with asymmetric composite free layer (ACFL) (MTJ embodiments 9-11 (FIGS. 9-11)).

TABLE 1

| | MTJ using PSA with DSF | MTJ using PSA with ACFL |
|---|---|---|
| Benefits | Smaller J_{c} | Higher E_{b} |
| | | Smaller J_{c} |
| | | No Intermediate States |
| Design | Insertion Material: | Insertion Material: |
| aspects | Weak coupling | Strong coupling |
| | PMA is optional | PMA is optional |
| | STT is important | No STT |
| | Thickness: | Thickness: |
| | “Top” FL is thinner | “Bottom” FL layer is thinner |

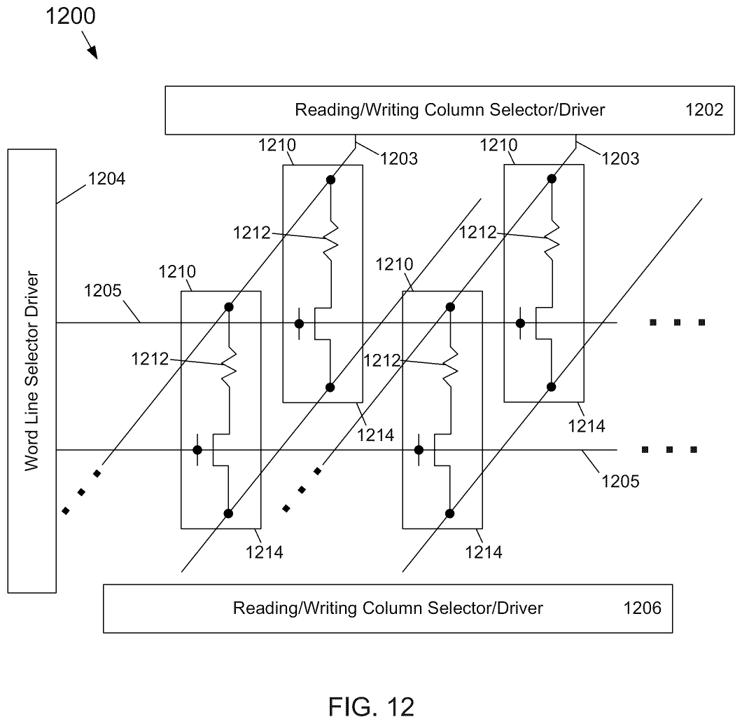

FIG. 12 depicts an exemplary embodiment of a memory 1200 that may use one or more of the magnetic junction bit structures 100-1100 disclosed herein. The magnetic memory 1200 includes reading/writing column select drivers 1202 and 1206 as well as a word line select driver 1204. Other and/or different components not shown may also be provided. The storage region of the memory 1200 includes magnetic storage cells 1210. Each magnetic storage cell includes at least one magnetic junction 1212 and at least one selection device 1214. In some embodiments, the selection device 1214 may be a transistor. The magnetic junctions 1212 may be one of the MTJ bit structures 100-1100 disclosed herein. Although one magnetic junction 1212 is shown per cell 1210, in other embodiments, another number of magnetic junctions 1212 may be provided per cell. As such, the magnetic memory 1200 may provide the benefits described herein.

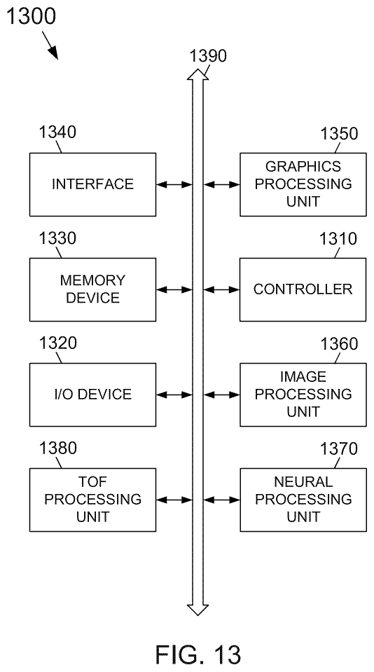

FIG. 13 depicts an electronic device 1300 that may include a memory device that includes magnetic tunnel junction bit structures according to the subject matter disclosed herein. Electronic device 1300 and the various system components of electronic device 1300 may be formed from one or modules. The electronic device 1300 may include a controller (or CPU) 1310, an input/output device 1320 such as, but not limited to, a keypad, a keyboard, a display, a touch-screen display, a 2D image sensor, a 3D image sensor, a memory 1330, an interface 1340, a GPU 1350, an imaging-processing unit 1360, a neural processing unit 1370, a TOF processing unit 1380 that are coupled to each other through a bus 1390. In one embodiment, the memory 1330 may include one or more magnetic tunnel junction bit structures according to the subject matter disclosed herein. In one embodiment, the 2D image sensor and/or the 3D image sensor may be part of the imaging processing unit 1360. In another embodiment, the 3D image sensor may be part of the TOF processing unit 1380. The controller 1310 may include, for example, at least one microprocessor, at least one digital signal processor, at least one microcontroller, or the like. The memory 1330 may be configured to store a command code to be used by the controller 1310 and/or to store a user data.

The interface 1340 may be configured to include a wireless interface that is configured to transmit data to or receive data from, for example, a wireless communication network using a RF signal. The wireless interface 1340 may include, for example, an antenna. The electronic system 1300 also may be used in a communication interface protocol of a communication system, such as, but not limited to, Code Division Multiple Access (CDMA), Global System for Mobile Communications (GSM), North American Digital Communications (NADC), Extended Time Division Multiple Access (E-TDMA), Wideband CDMA (WCDMA), CDMA2000, Wi-Fi, Municipal Wi-Fi (Muni Wi-Fi), Bluetooth, Digital Enhanced Cordless Telecommunications (DECT), Wireless Universal Serial Bus (Wireless USB), Fast low-latency access with seamless handoff Orthogonal Frequency Division Multiplexing (Flash-OFDM), IEEE 802.20, General Packet Radio Service (GPRS), iBurst, Wireless Broadband (WiBro), WiMAX, WiMAX-Advanced, Universal Mobile Telecommunication Service-Time Division Duplex (UMTS-TDD), High Speed Packet Access (HSPA), Evolution Data Optimized (EVDO), Long Term Evolution-Advanced (LTE-Advanced), Multichannel Multipoint Distribution Service (MMDS), Fifth-Generation Wireless (5G), Sixth-Generation Wireless (6G), and so forth.

Embodiments of the subject matter and the operations described in this specification may be implemented in digital electronic circuitry, or in computer software, firmware, or hardware, including the structures disclosed in this specification and their structural equivalents, or in combinations of one or more of them. Embodiments of the subject matter described in this specification may be implemented as one or more computer programs, i.e., one or more modules of computer-program instructions, encoded on computer-storage medium for execution by, or to control the operation of data-processing apparatus. Alternatively or additionally, the program instructions can be encoded on an artificially-generated propagated signal, e.g., a machine-generated electrical, optical, or electromagnetic signal, which is generated to encode information for transmission to suitable receiver apparatus for execution by a data processing apparatus. A computer-storage medium can be, or be included in, a computer-readable storage device, a computer-readable storage substrate, a random or serial-access memory array or device, or a combination thereof. Moreover, while a computer-storage medium is not a propagated signal, a computer-storage medium may be a source or destination of computer-program instructions encoded in an artificially-generated propagated signal. The computer-storage medium can also be, or be included in, one or more separate physical components or media (e.g., multiple CDs, disks, or other storage devices). Additionally, the operations described in this specification may be implemented as operations performed by a data-processing apparatus on data stored on one or more computer-readable storage devices or received from other sources.

While this specification may contain many specific implementation details, the implementation details should not be construed as limitations on the scope of any claimed subject matter, but rather be construed as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described herein. Other embodiments are within the scope of the following claims. In some cases, the actions set forth in the claims may be performed in a different order and still achieve desirable results. Additionally, the processes depicted in the accompanying figures do not necessarily require the particular order shown, or sequential order, to achieve desirable results. In certain implementations, multitasking and parallel processing may be advantageous.

As will be recognized by those skilled in the art, the innovative concepts described herein may be modified and varied over a wide range of applications. Accordingly, the scope of claimed subject matter should not be limited to any of the specific exemplary teachings discussed above, but is instead defined by the following claims.

What is claimed is:

1. A memory device, comprising:
a magnetic tunnel junction (MTJ) structure comprising perpendicular shape anisotropy, and the MTJ structure further comprising:
a reference layer comprising a first side and a second side that is opposite the first side of the reference layer;
a non-magnetic spacer comprising a first side and a second side, the first side of the non-magnetic spacer being on the second side of the reference layer; and
free layer comprising a first side and a second side, the first side of the free layer being on the second side of the non-magnetic spacer, the free layer further comprising a first layer on the first side of the free layer, a second layer on the second side of the free layer and a coupling layer disposed between the first layer and the second layer, a ratio of a saturation magnetization Ms_{FL2} of the second layer to a saturation magnetization Ms_{FL1} of the first layer ranging from 0.2 to 0.8 inclusive.

2. The memory device of claim 1, wherein a thickness of the first layer is substantially equal to a thickness of the second layer.

3. The memory device of claim 1, wherein a ratio of a thickness of the free layer to a critical dimension of the MTJ structure is between 0.5 and 5.0 inclusive, and
wherein the critical dimension of the MTJ structure ranges from 4-22 nm inclusive.

4. The memory device of claim 1, wherein the first layer comprises CoFeB, and
wherein the second layer comprises CoFeX or CoFeBX in which X is a diluent element comprising vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi).

5. The memory device of claim 1, wherein the coupling layer comprises a resistance area product ranging from 0.1 to 3.0 Ohm*μm² inclusive, and
wherein the coupling layer comprises a tunnel magnetoresistance (TMR) that is greater than 30%.

6. The memory device of claim 1, further comprising a capping layer on the second side of the free layer comprising one or more of magnesium oxide (MgO), tantalum oxide (TaO), niobium oxide (NiO), iridium oxide (IrO), sodium oxide (NaO), rhodium oxide (RhO), and osmium oxide (OsO).

7. The memory device of claim 1, wherein the first layer comprises a first side and a second side that is opposite the first side of the first layer, the first layer further comprising a first sublayer, a second sublayer and a first texture blocking layer disposed between the first sublayer and the second sublayer, the first sublayer being disposed distal to the coupling layer, and the second sublayer being disposed proximate to the coupling layer, and
wherein the second layer comprises a first side and a second side that is opposite the second side of the second layer, the second layer further comprising a third sublayer, a fourth sublayer and a second texture blocking layer disposed between the third sublayer and the fourth sublayer, the third sublayer being disposed proximate to the coupling layer and the fourth sublayer being disposed distal to the coupling layer.

8. The memory device of claim 7, wherein at least one of the second sublayer and the third sublayer comprises a CoFe or a Heusler material.

9. The memory device of claim 1, wherein the first layer comprises a first side and a second side that is opposite the first side of the first layer, the first layer further comprising a first sublayer and a second sublayer, the first sublayer being disposed proximate to the non-magnetic spacer, and the second sublayer being disposed distal to the non-magnetic spacer, and
wherein the reference layer further comprises a first layer on the first side of the reference layer and a second layer on the second side of the reference layer, the second layer of the reference layer comprising a third sublayer and a fourth sublayer, the third sublayer being distal to the non-magnetic spacer and the fourth sublayer being proximate to the non-magnetic spacer.

10. The memory device of claim 9, wherein at least one of the second sublayer and the third sublayer comprises a CoFe or a Heusler material.

11. The memory device of claim 1, further comprising an array of the MTJ structures.

12. A memory device, comprising:
a magnetic tunnel junction (MTJ) structure comprising perpendicular shape anisotropy, and the MTJ structure further comprising:
a reference layer comprising a first side and a second side that is opposite the first side of the reference layer;
a non-magnetic spacer comprising a first side and a second side, the first side of the non-magnetic spacer being on the second side of the reference layer; and
free layer comprising a first side and a second side, the first side of the free layer being on the second side of the non-magnetic spacer, the free layer further comprising a first layer on the first side of the free layer, a second layer on the second side of the free layer and a coupling layer disposed between the first layer and the second layer, a saturation magnetization Ms_{FL2} of the second layer being substantially equal to a saturation magnetization Ms_{FL1} of the first layer, and a ratio of a thickness of the first layer to a thickness of the second layer ranging from 2 to 4 inclusive.

13. The memory device of claim 12, wherein a ratio of a thickness of the free layer to a critical dimension of the MTJ structure is between 0.5 and 5.0 inclusive, and
wherein the critical dimension of the MTJ structure ranges from 4-22 nm inclusive.

14. The memory device of claim 12, wherein the first layer comprises CoFeB, and
wherein the second layer comprises CoFeX or CoFeBX in which X is a diluent element comprising vanadium (V), molybdenum (Mo), titanium (Ti), zirconium (Zr), hafnium (Hf), aluminum (Al), niobium (Nb), tantalum (Ta), chromium (Cr), rhodium (Rh) or bismuth (Bi).

15. The memory device of claim 12, wherein the coupling layer comprises a resistance area product ranging from 0.1 to 3.0 Ohm*μm² inclusive, and
wherein the coupling layer comprises a tunnel magnetoresistance (TMR) that is greater than 30%.

16. The memory device of claim 12, further comprising a capping layer on the second side of the free layer comprising one or more of magnesium oxide (MgO), tantalum oxide (TaO), niobium oxide (NiO), iridium oxide (IrO), sodium oxide (NaO), rhodium oxide (RhO), or osmium oxide (OsO).

17. A memory device, comprising:
a magnetic tunnel junction (MTJ) structure comprising perpendicular shape anisotropy, and the MTJ structure further comprising:
a reference layer comprising a first side and a second side that is opposite the first side of the reference layer;
a non-magnetic spacer comprising a first side and a second side, the first side of the non-magnetic spacer being on the second side of the reference layer; and
free layer comprising a first side and a second side, the first side of the free layer being on the second side of the non-magnetic spacer, the free layer further comprising a first layer on the first side of the free layer, a second layer on the second side of the free layer and a coupling layer disposed between the first layer and the second layer, a saturation magnetization Ms_{FL2} of the second layer being substantially equal to a saturation magnetization Ms_{FL1} of the first layer, and a ratio of a thickness of the first layer to a thickness of the second layer ranging from 2 to 4 inclusive,
the first layer comprising a first side and a second side that is opposite the first side of the first layer, the first layer further comprising a first sublayer, a second sublayer and a first texture blocking layer disposed between the first sublayer and the second sublayer, the first sublayer being disposed distal to the coupling layer, and the second sublayer being disposed proximate to the coupling layer, and
the second layer comprising a first side and a second side that is opposite the second side of the second layer, the second layer further comprising a third sublayer, a fourth sublayer and a second texture blocking layer disposed between the third sublayer and the fourth sublayer, the third sublayer being disposed proximate to the coupling layer and the fourth sublayer being disposed distal to the coupling layer, at least one of the second sublayer and the third sublayer comprises a CoFe or a Heusler material.

18. The memory device of claim 17, wherein a ratio of a thickness of the free layer to a critical dimension of the MTJ structure is between 0.5 and 5.0 inclusive, and
wherein the critical dimension of the MTJ structure ranges from 4-22 nm inclusive.

19. A memory device, comprising:
a magnetic tunnel junction (MTJ) structure comprising perpendicular shape anisotropy, and the MTJ structure further comprising:
a reference layer comprising a first side and a second side that is opposite the first side of the reference layer, the reference layer further comprises a first layer on the first side of the reference layer and a second layer on the second side of the reference layer, the second layer of the reference layer comprising a first sublayer and a second sublayer, and the second sublayer being distal to the first side of the reference layer and proximate to the second side of the reference layer;
a non-magnetic spacer comprising a first side and a second side, the first side of the non-magnetic spacer being on the second side of the reference layer; and
free layer comprising a first side and a second side, the first side of the free layer being on the second side of the non-magnetic spacer, the free layer further comprising a first layer on the first side of the free layer, a second layer on the second side of the free layer and a coupling layer disposed between the first layer and the second layer, a saturation magnetization Ms_{FL2} of the second layer being substantially equal to a saturation magnetization Ms_{FL1} of the first layer, and a ratio of a thickness of the first layer to a thickness of the second layer ranging from 2 to 4 inclusive,
the first layer comprising a first side and a second side that is opposite the first side of the first layer, the first layer further comprising a third sublayer and a fourth sublayer, the third sublayer being disposed proximate to the non-magnetic spacer, and the fourth sublayer being disposed distal to the non-magnetic spacer, at least one of the second sublayer and the third sublayer comprises a CoFe or a Heusler material.

20. The memory device of claim 19, wherein a ratio of a thickness of the free layer to a critical dimension of the MTJ structure is between 0.5 and 5.0 inclusive, and
wherein the critical dimension of the MTJ structure ranges from 4-22 nm inclusive.
